(19) United States
(12) Patent Application Publication
(10) Pub. No.: US 2020/0339537 A1
(43) Pub. Date: Oct. 29, 2020

(54) NOVEL COMPOUND AS INHIBITOR AGAINST BINDING OF PF34 PROTEIN TO NEDD4-1 PROTEIN AND USE THEREOF

(71) Applicant: ENHANCEDBIO INC., Seoul (KR)

(72) Inventor: Doo Seop KIM, Seoul (KR)

(21) Appl. No.: 16/767,796

(22) PCT Filed: Nov. 30, 2018
(86) PCT No.: PCT/KR2018/015011

§ 371 Date: May 28, 2020

(30) Foreign Application Priority Data
Nov. 30, 2017 (KR) 10-2017-0162752

(51) Int. Cl. C07D 401/14; A61P 35/04; C07D 417/14; C07D 487/04; C07D 498/22; C07D 403/12; C07D 209/08; C07D 401/12; C07D 417/12

(52) U.S. Cl. CPC C07D 401/14; A61P 35/04; C07D 417/14; C07D 487/04; C07D 417/12; C07D 403/12; C07D 209/08; C07D 401/12; C07D 498/22

(57) ABSTRACT

The present invention relates to a novel compound having an inhibitory effect on binding between p34 protein and NEDD4-1 protein. A pharmaceutical composition comprising the compound of the present invention as an active ingredient can be effectively used to treat and/or prevent cancer.

### TECHNICAL FIELD

The present invention relates to a novel compound and a use thereof, and more particularly to a novel compound having an inhibitory effect on binding between p34 protein and NEDD4-1 protein, or a pharmaceutically acceptable salt or solvate thereof, a pharmaceutical composition comprising such a compound as an active ingredient, and uses thereof.

### BACKGROUND ART

Cancer is one of the most serious diseases that threaten human health and is a disease that occurs when cells have undergone a series of mutations so that they proliferate in an unlimited and uncontrolled manner and eventually become immortalized. Causes of cancer may include environmental or external factors such as chemical substances, viruses, bacteria, and ionizing radiation, and internal factors such as congenital genetic mutations (Klaunig & Kamendulis, Annu Rev Pharmacol Toxicol., 44:239-267, 2004). When cancers are found in their early stage, there are treatments such as surgery, radiation therapy, and chemotherapy; however, adverse effects resulting therefrom are emerging as major problems. For terminal cancers or metastatic cancers, there are no special treatments; and patients with such cancers just live for a given limited period of time until death.

As of 2011, regarding the mortality rates per 100,000 people for six major cancers, colorectal cancer was 15.4%. The incidence rate of colorectal cancer per 100,000 people was increasing from 21.8% to 50.3%. More specifically, it was identified that the expression of PTEN decreases when colorectal cancer develops and metastasizes, during which the role of PTEN is said to be very important. Based on this, it can be said that it is necessary to use PTEN as an endpoint when developing anticancer drugs. Once PTEN is activated, it inhibits cell cycle progression and induces apoptosis.

In addition, tumor growth is inhibited in a case where PTEN is overexpressed in an animal model derived from a colorectal cancer cell line. It is identifiable that tumor growth is remarkably inhibited in a case where PTEN is overexpressed in animal models derived from the colon cancer cell lines SW480 and HT29.

It has been reported that PTEN, which functions as a tumor suppressor in PI3K/PTEN signaling in colorectal cancer, is rarely expressed in colorectal cancer. It is reported that among colorectal cancer patients, 75.4% of the patients with liver metastasis have little expression of PTEN and their 5-year survival rate is greatly decreased.

Such patients, more specifically, 50% to 60% of the patients, including those with metastatic colorectal cancer who have relapsed at a rate of 20% to 50% even after radical resection for colorectal cancer, are targeted for anticancer drug therapy for the treatment of colorectal cancer. Recently, biological targeted therapeutics have been applied to anticancer therapy for colorectal cancer and have shown a therapeutic effect in some of patients with metastatic colorectal cancer. However, the therapeutic effect is insufficient.

### Technical Problem

An object of the present invention is to provide a compound capable of selectively and effectively inhibiting binding between p34 protein and NEDD4-1 protein, or a pharmaceutically acceptable salt thereof.

Another object of the present invention is to provide a pharmaceutical composition comprising the compound as an active ingredient.

Yet another object of the present invention is to provide a pharmaceutical composition capable of preventing or treating various diseases mediated by binding between p34 protein and NEDD4-1 protein, in which the pharmaceutical composition comprises the compound as an active ingredient and thus inhibits the binding between p34 protein and NEDD4-1 protein in a selective and effective manner.

### Solution to Problem

In order to achieve the above objects, the present invention provides a compound represented by Formula 1 or a pharmaceutically acceptable salt thereof:

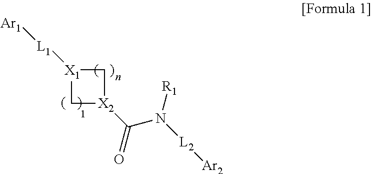

in the formula,

l and n are each independently an integer from 1 to 5,

X₁ and X₂ are each independently N or CH, and at least one thereof is N,

L₁ and L₂ are the same or different from each other and are each independently a single bond, a carbonyl group, an unsubstituted or substituted alkylene group having 1 to 10 carbon atoms, or an unsubstituted or substituted arylene group having 6 to 30 carbon atoms,

Ar₁ and Ar₂ are the same or different from each other and are each independently selected from the group consisting of an unsubstituted or substituted aryl group having 6 to 30 carbon atoms, an unsubstituted or substituted aralkyl group having 6 to 30 carbon atoms, an unsubstituted or substituted heteroaryl group having 3 to 30 carbon atoms, an unsubstituted or substituted heteroarylalkyl group having 6 to 30 carbon atoms, an unsubstituted or substituted cycloalkyl group having 3 to 40 carbon atoms, and an unsubstituted or substituted heterocycloalkyl group having 3 to 40 carbon atoms, and

R₁ is selected from the group consisting of hydrogen, deuterium, a hydroxy group, an alkyl group having 1 to 30 carbon atoms, a cycloalkyl group having 1 to 20 carbon atoms, an alkenyl group having 2 to 30 carbon atoms, an alkynyl group having 2 to 24 carbon atoms, an aralkyl group having 7 to 30 carbon atoms, and an aryl group having 6 to 30 carbon atoms,

wherein the substituted alkylene group, substituted arylene group, substituted heteroarylene group, substituted aryl group, substituted aralkyl group, substituted heteroaryl group, substituted heteroarylalkyl group, substituted cycloalkyl group, and substituted heterocycloalkyl group are those groups each independently obtained by being substituted with at least one substituent selected from the group consisting of hydrogen, deuterium, a cyano group, a nitro group, a halogen group, a hydroxy group, an alkyl group having 1 to 30 carbon atoms, a cycloalkyl group having 1 to 20 carbon atoms, an alkenyl group having 2 to 30 carbon atoms, an alkynyl group having 2 to 24 carbon atoms, an aralkyl group having 7 to 30 carbon atoms, an aryl group having 6 to 30 carbon atoms, a heteroaryl group having 6 to 30 carbon atoms, a heteroaralkyl group having 3 to 30 carbon atoms, an alkoxy group having 1 to 30 carbon atoms, an alkylamino group having 1 to 30 carbon atoms, an arylamino group having 6 to 30 carbon atoms, an aralkylamino group having 6 to 30 carbon atoms, a heteroarylamino group having 6 to 30 carbon atoms, an alkylsilyl group having 1 to 30 carbon atoms, a cycloalkyl group having 3 to 40 carbon atoms, a heterocycloalkyl group having 3 to 40 carbon atoms, an arylsilyl group having 6 to 60 carbon atoms, and an aryloxy group having 6 to 30 carbon atoms; and in a case where those groups are substituted with a plurality of substituents, the substituents are the same or different from each other and may combine with an adjacent group to form a substituted or unsubstituted ring.

According to another aspect of the present invention, there is provided a use of a pharmaceutical composition for prevention or treatment of a disease mediated by simultaneous expression of p34 and NEDD4-1, the pharmaceutical composition comprising, as an active ingredient, a compound of Formula 1 or a pharmaceutically acceptable salt thereof.

It is particularly preferred that the disease is selected from tumor cells (for example, carcinomas of breast, colorectum, and colon).

### Advantageous Effects of Invention

The novel compound according to the present invention can selectively or simultaneously inhibit various diseases mediated by simultaneous expression of p34 and NEDD4-1. Thus, the novel derivatives according to the present invention can be effectively used for treatment or prevention of tumor cells (for example, carcinomas of breast, colorectum, and colon).

### BRIEF DESCRIPTION OF DRAWINGS

FIGS. 1A to 1D illustrate graphs showing results obtained by performing NMR titration to determine a site of NEDD4-1 protein where binding to p34 is made.

### DETAILED DESCRIPTION OF INVENTION

The definitions listed below are definitions of various terms used to describe the present invention. Unless specified otherwise, each of these definitions applies, throughout the specification, individually or as part of a term that includes such a definition.

As used herein, the term ‘halogen’, unless stated otherwise, refers to fluorine, chlorine, bromine, or iodine, or any one among all these elements.

As used herein, the term ‘alkyl’, unless otherwise stated, refers to a saturated, linear or branched hydrocarbon radical represented by CₙH₂ₙ₊₁, and specifically to a saturated, linear or branched hydrocarbon radical that contains 1 to 6, 1 to 8, 1 to 10, or 1 to 20 carbon atoms, respectively. Examples of these radicals include, but are not limited to, methyl, ethyl, propyl, isopropyl, n-butyl, tert-butyl, neopentyl, n-hexyl, heptyl, and octyl radicals.

As used herein, the term ‘alkenyl’, unless stated otherwise, refers to a monovalent group derived from an unsaturated, linear or branched hydrocarbon moiety having at least one carbon-carbon double bond, and specifically to an unsaturated, linear or branched monovalent group that contains 2 to 6, 2 to 8, 2 to 10, or 2 to 20 carbon atoms, respectively. Examples thereof include, but are not limited to, ethenyl, propenyl, butenyl, 1-methyl-2-buten-1-yl, heptenyl, and octenyl radicals.

As used herein, the term ‘cycloalkyl’, unless stated otherwise, refers to a monovalent group derived from a monocyclic or polycyclic saturated or partially unsaturated carbocyclic ring compound. Examples of C3-C8-cycloalkyl include, but not limited to, cyclopropyl, cyclobutyl, cyclopentyl, cyclohexyl, cycloheptyl, and cyclooctyl; and examples of C3-C12-cycloalkyl include, but not limited to, cyclopropyl, cyclobutyl, cyclopentyl, cyclohexyl, bicyclo[2.2.1]heptyl, and bicyclo[2.2.2]octyl. Also contemplated is a monovalent group derived by removal of a single hydrogen atom from a monocyclic or polycyclic carbocyclic ring compound having at least one carbon-carbon double bond. Examples of this group include, but are not limited to, cyclopropenyl, cyclobutenyl, cyclopentenyl, cyclohexenyl, cycloheptenyl, cyclooctenyl, and the like.

As used herein, the term ‘cycloalkenyl’, unless stated otherwise, refers to a partially unsaturated carbocyclic ring containing 3 to 6 carbon atoms and having a carbon-carbon double bond therein. Examples of this group include, but are not limited to, cyclopentenyl and cyclohexenyl.

As used herein, the term ‘aryl’, unless stated otherwise, refers to a mono- or poly-cyclic carbocyclic ring system having one or more fused or non-fused aromatic rings. Examples thereof include, but are not limited to, phenyl, naphthyl, tetrahydronaphthyl, indenyl, idenyl, and the like.

As used herein, the term ‘heterocycloalkyl’, unless stated otherwise, refers to a saturated or partially unsaturated 3- to 10-membered monocyclic or polycyclic substituent, containing one or more, for example, 1 to 4 heteroatoms selected from N, O, and S. Examples of monocyclic heterocycloalkyl include, but are not limited to, piperidinyl, morpholinyl, thiamorpholinyl, pyrrolidinyl, imidazolidinyl, tetrahydrofuranyl, piperazinyl, and similar groups thereto.

As used herein, the term ‘heteroaryl’, unless stated otherwise, refers to a 5- to 12-membered monocyclic, bicyclic, or higher-cyclic aromatic group, containing one or more, for example, 1 to 4 heteroatoms selected from N, O, and S. Examples of monocyclic heteroaryl include, but are not limited to, thiazolyl, oxazolyl, thiophenyl, furanyl, pyrrolyl, imidazolyl, isoxazolyl, pyrazolyl, triazolyl, thiadiazolyl, tetrazolyl, oxadiazolyl, pyridinyl, pyridazinyl, pyrimidinyl, pyrazinyl, and similar groups thereto. Examples of bicyclic heteroaryl include, but are not limited to, indolyl, benzothiophenyl, benzofuranyl, benzimidazolyl, benzoxazolyl, benzisoxazolyl, benzthiazolyl, benzthiadiazolyl, benztriazolyl, quinolinyl, isoquinolinyl, furinyl, furopyridinyl, and similar groups thereto.

As used herein, the term ‘non-aromatic condensed heteropolycycle’ refers to a group (for example, having 5 to 10 nuclear atoms) in which two or more rings are condensed with each other and a heteroatom selected from N, O, and S other than carbon is included as a ring-forming atom, and in which the entire molecule has non-aromaticity. Examples of the non-aromatic condensed heteropolycycle may include, but are not limited to, benzo[d][1,3]dioxole and the like.

Hereinafter, the present invention will be described in more detail.

The present invention provides a compound of Formula 1 or a pharmaceutically acceptable salt thereof:

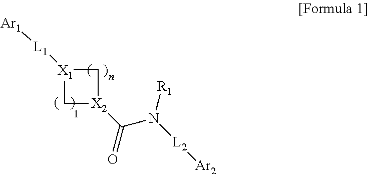

in the formula,

l and n are each independently an integer from 1 to 5,

X₁ and X₂ are each independently N or CH, and at least one thereof is N,

L₁ and L₂ are the same or different from each other and are each independently a single bond, a carbonyl group, an unsubstituted or substituted alkylene group having 1 to 10 carbon atoms, or an unsubstituted or substituted arylene group having 6 to 30 carbon atoms,

Ar₁ and Ar₂ are the same or different from each other and are each independently selected from the group consisting of an unsubstituted or substituted aryl group having 6 to 30 carbon atoms, an unsubstituted or substituted aralkyl group having 6 to 30 carbon atoms, an unsubstituted or substituted heteroaryl group having 3 to 30 carbon atoms, an unsubstituted or substituted heteroarylalkyl group having 6 to 30 carbon atoms, an unsubstituted or substituted cycloalkyl group having 3 to 40 carbon atoms, and an unsubstituted or substituted heterocycloalkyl group having 3 to 40 carbon atoms, and

R₁ is selected from the group consisting of hydrogen, deuterium, a hydroxy group, an alkyl group having 1 to 30 carbon atoms, a cycloalkyl group having 1 to 20 carbon atoms, an alkenyl group having 2 to 30 carbon atoms, an alkynyl group having 2 to 24 carbon atoms, an aralkyl group having 7 to 30 carbon atoms, and an aryl group having 6 to 30 carbon atoms,

wherein the substituted alkylene group, substituted arylene group, substituted heteroarylene group, substituted aryl group, substituted aralkyl group, substituted heteroaryl group, substituted heteroarylalkyl group, substituted cycloalkyl group, and substituted heterocycloalkyl group are those groups each independently obtained by being substituted with at least one substituent selected from the group consisting of hydrogen, deuterium, a cyano group, a nitro group, a halogen group, a hydroxy group, an alkyl group having 1 to 30 carbon atoms, a cycloalkyl group having 1 to 20 carbon atoms, an alkenyl group having 2 to 30 carbon atoms, an alkynyl group having 2 to 24 carbon atoms, an aralkyl group having 7 to 30 carbon atoms, an aryl group having 6 to 30 carbon atoms, a heteroaryl group having 6 to 30 carbon atoms, a heteroaralkyl group having 3 to 30 carbon atoms, an alkoxy group having 1 to 30 carbon atoms, an alkylamino group having 1 to 30 carbon atoms, an arylamino group having 6 to 30 carbon atoms, an aralkylamino group having 6 to 30 carbon atoms, a heteroarylamino group having 6 to 30 carbon atoms, an alkylsilyl group having 1 to 30 carbon atoms, a cycloalkyl group having 3 to 40 carbon atoms, a heterocycloalkyl group having 3 to 40 carbon atoms, an arylsilyl group having 6 to 60 carbon atoms, and an aryloxy group having 6 to 30 carbon atoms; and in a case where those groups are substituted with a plurality of substituents, the substituents are the same or different from each other and may combine with an adjacent group to form a substituted or unsubstituted ring.

In a specific embodiment of the present invention, the compound is preferably a compound in which l is 1 or 2.

In a specific embodiment of the present invention, the compound is preferably a compound in which n is 1 or 2.

In a specific embodiment of the present invention, Ar₁ may be selected from the group consisting of compounds represented by Formulas 2 to 4:

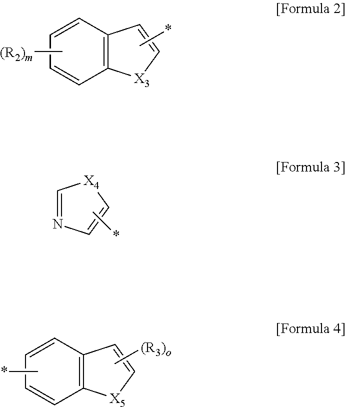

in the formulas,

* means a site where a bond is formed,

m is an integer from 0 to 4,

o is an integer from 0 to 2,

X₃ to X₅ are the same or different from each other and are each independently selected from the group consisting of N(R₄), S, O, and C(R₅)(R₆),

R₂ to R₆ are the same or different from each other and are each independently selected from the group consisting of hydrogen, deuterium, a cyano group, a nitro group, a halogen group, a hydroxy group, an alkyl group having 1 to 30 carbon atoms, a cycloalkyl group having 1 to 20 carbon atoms, an alkenyl group having 2 to 30 carbon atoms, an alkynyl group having 2 to 24 carbon atoms, an aralkyl group having 7 to 30 carbon atoms, an aryl group having 6 to 30 carbon atoms, a heteroaryl group having 6 to 30 carbon atoms, a heteroaralkyl group having 3 to 30 carbon atoms, an alkoxy group having 1 to 30 carbon atoms, an alkylamino group having 1 to 30 carbon atoms, an arylamino group having 6 to 30 carbon atoms, an aralkylamino group having 6 to 30 carbon atoms, a heteroarylamino group having 6 to 30 carbon atoms, an alkylsilyl group having 1 to 30 carbon atoms, a cycloalkyl group having 3 to 40 carbon atoms, a heterocycloalkyl group having 3 to 40 carbon atoms, an arylsilyl group having 6 to 60 carbon atoms, and an aryloxy group having 6 to 30 carbon atoms.

In a specific embodiment of the present invention, Ar₂ may be selected from the group consisting of compounds represented by Formulas 5 to 7:

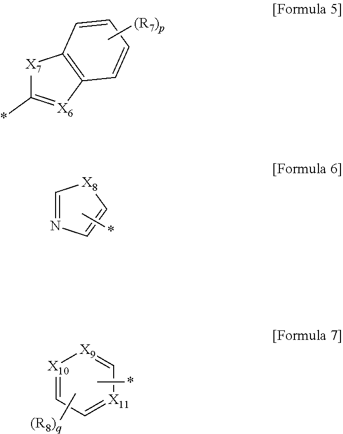

in the formulas,

p is an integer from 0 to 4,

q is an integer from 0 to 2,

X₆ and X₉ to X₁₁ are the same or different from each other and are each independently selected from the group consisting of N, O, S, and C(R₉),

X₇ and X₈ are the same or different from each other and are each independently selected from the group consisting of N(R₁₀), O, S, and C(R₁₁)(R₁₂),

R₆ to R₁₂ are the same or different from each other and are each independently selected from the group consisting of hydrogen, deuterium, a cyano group, a nitro group, a halogen group, a hydroxy group, an alkyl group having 1 to 30 carbon atoms, a cycloalkyl group having 1 to 20 carbon atoms, an alkenyl group having 2 to 30 carbon atoms, an alkynyl group having 2 to 24 carbon atoms, an aralkyl group having 7 to 30 carbon atoms, an aryl group having 6 to 30 carbon atoms, a heteroaryl group having 6 to 30 carbon atoms, a heteroaralkyl group having 3 to 30 carbon atoms, an alkoxy group having 1 to 30 carbon atoms, an alkylamino group having 1 to 30 carbon atoms, an arylamino group having 6 to 30 carbon atoms, an aralkylamino group having 6 to 30 carbon atoms, a heteroarylamino group having 6 to 30 carbon atoms, an alkylsilyl group having 1 to 30 carbon atoms, a cycloalkyl group having 3 to 40 carbon atoms, a heterocycloalkyl group having 3 to 40 carbon atoms, an arylsilyl group having 6 to 60 carbon atoms, and an aryloxy group having 6 to 30 carbon atoms.

In a specific embodiment of the present invention, the compound of Formula 1 may be selected from, but is not limited to, the group consisting of the following compounds:

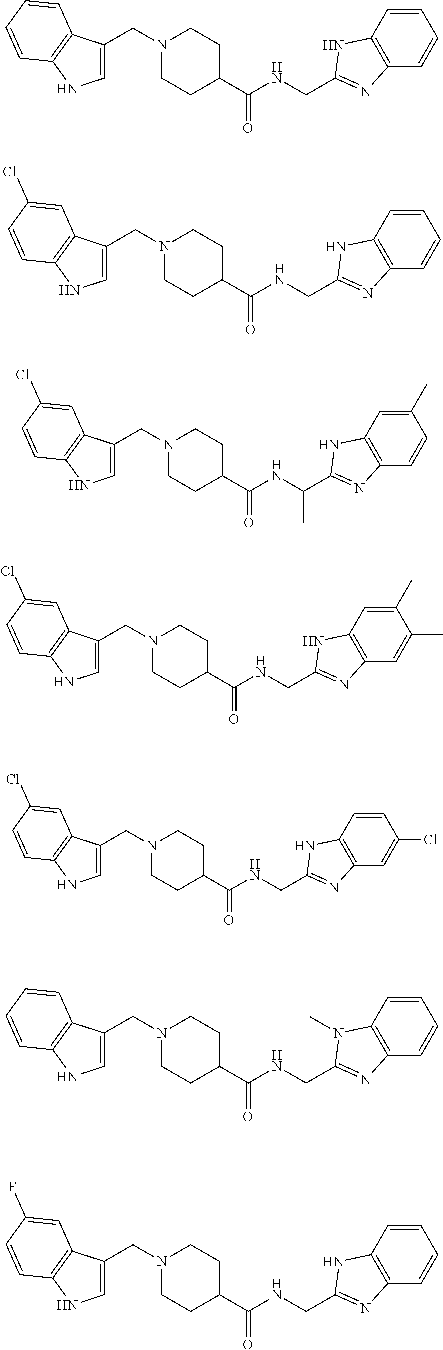

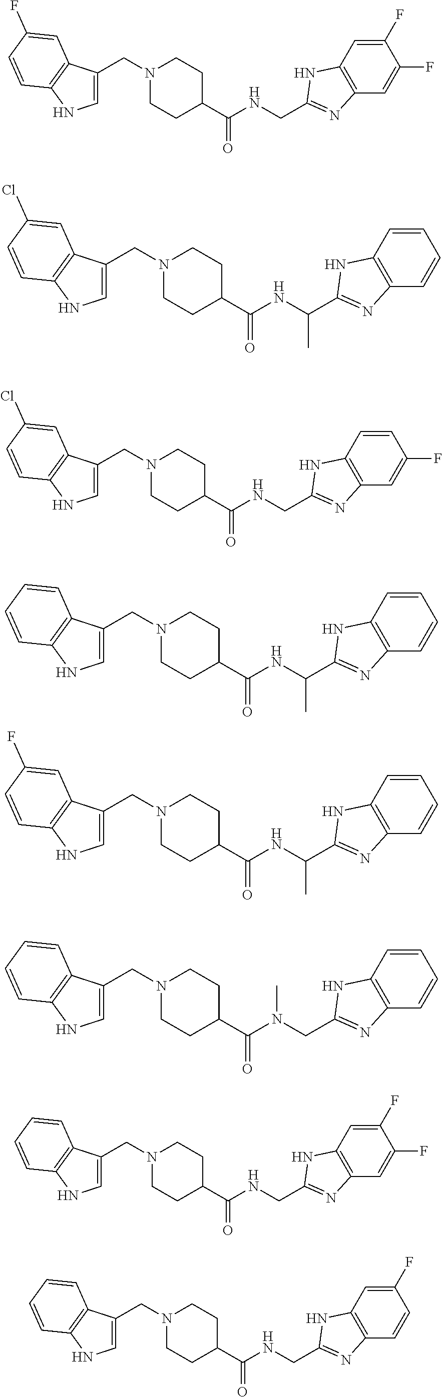

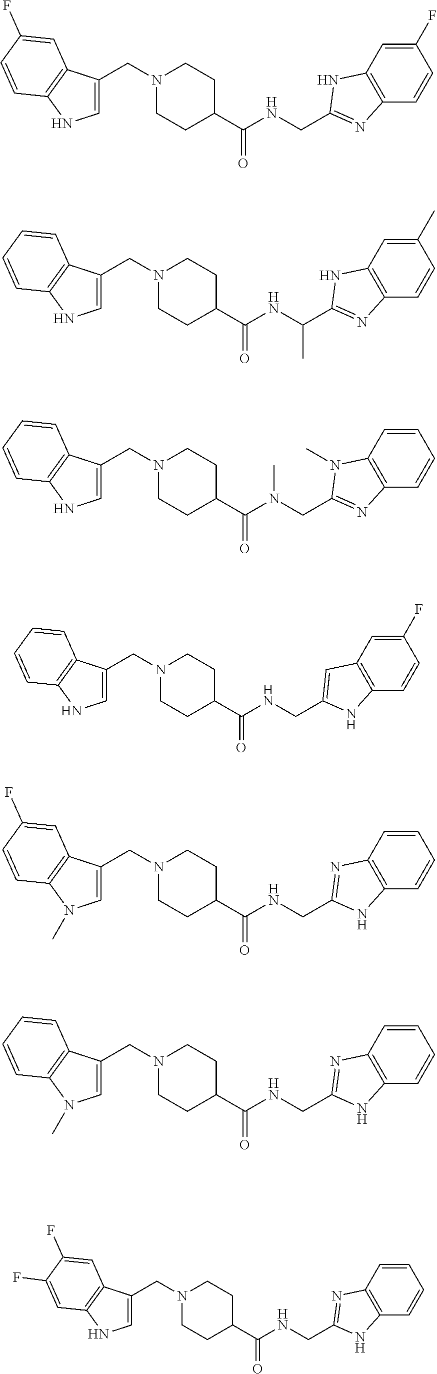

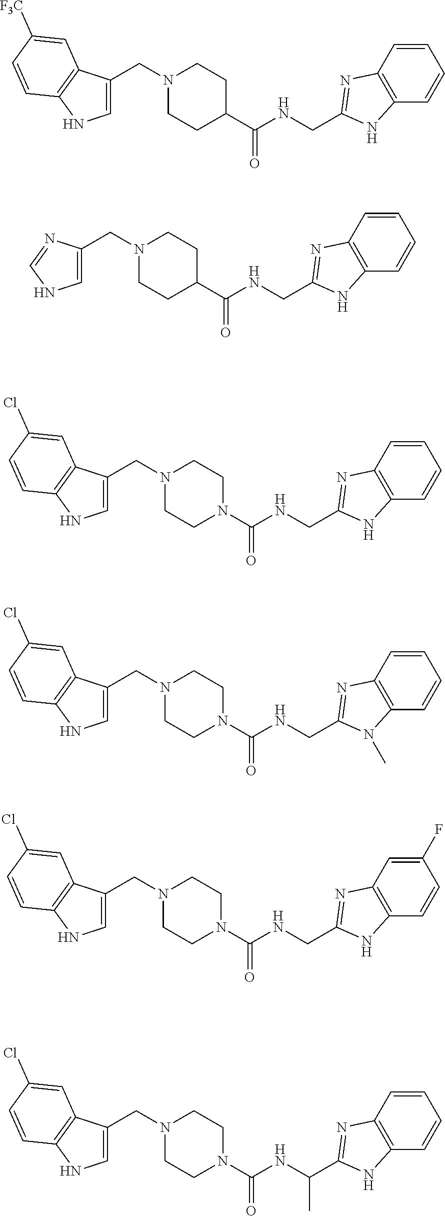 [SMILES: O=C(NCc1nc2ccccc2[nH]1)C1CCN(Cc2c[nH]c3ccc(C(F)(F)F)cc23)CC1 ; O=C(NCc1nc2ccccc2[nH]1)C1CCN(Cc2c[nH]cn2)CC1 ; O=C(NCc1nc2ccccc2[nH]1)N1CCN(Cc2c[nH]c3ccc(Cl)cc23)CC1 ; Cn1c(CNC(=O)N2CCN(Cc3c[nH]c4ccc(Cl)cc34)CC2)nc2ccccc21 ; O=C(NCc1nc2cc(F)ccc2[nH]1)N1CCN(Cc2c[nH]c3ccc(Cl)cc23)CC1 ; CC(NC(=O)N1CCN(Cc2c[nH]c3ccc(Cl)cc23)CC1)c1nc2ccccc2[nH]1]

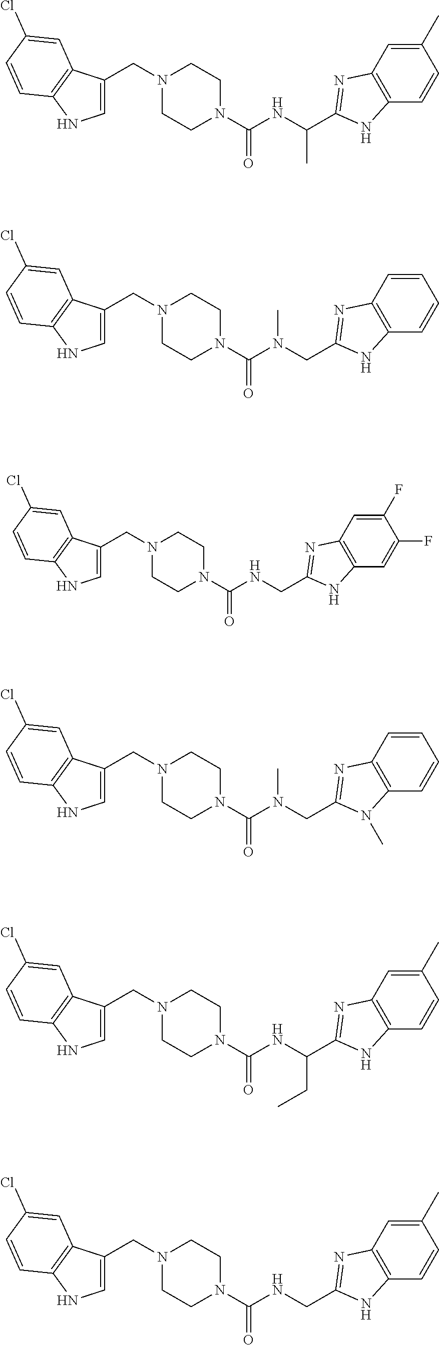 [SMILES: Cc1ccc2[nH]c(C(C)NC(=O)N3CCN(Cc4c[nH]c5ccc(Cl)cc45)CC3)nc2c1 ; CN(Cc1nc2ccccc2[nH]1)C(=O)N1CCN(Cc2c[nH]c3ccc(Cl)cc23)CC1 ; O=C(NCc1nc2cc(F)c(F)cc2[nH]1)N1CCN(Cc2c[nH]c3ccc(Cl)cc23)CC1 ; CN(Cc1nc2ccccc2n1C)C(=O)N1CCN(Cc2c[nH]c3ccc(Cl)cc23)CC1 ; CCC(NC(=O)N1CCN(Cc2c[nH]c3ccc(Cl)cc23)CC1)c1nc2cc(C)ccc2[nH]1 ; Cc1ccc2[nH]c(CNC(=O)N3CCN(Cc4c[nH]c5ccc(Cl)cc45)CC3)nc2c1]

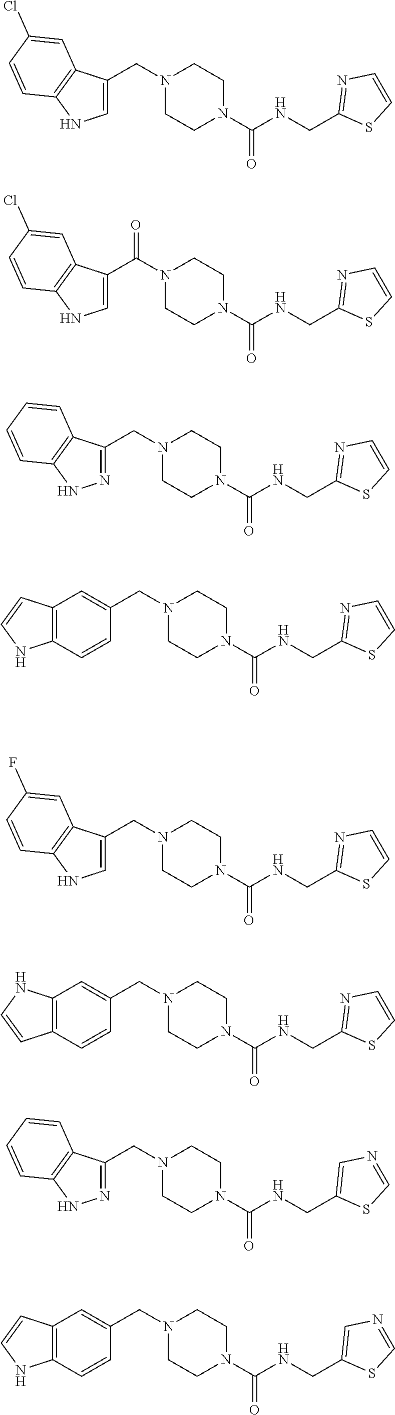

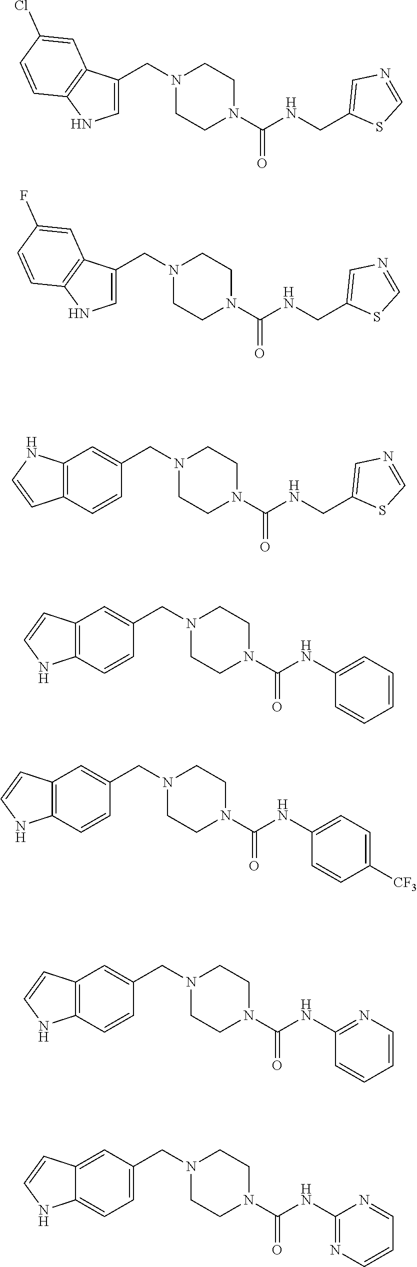

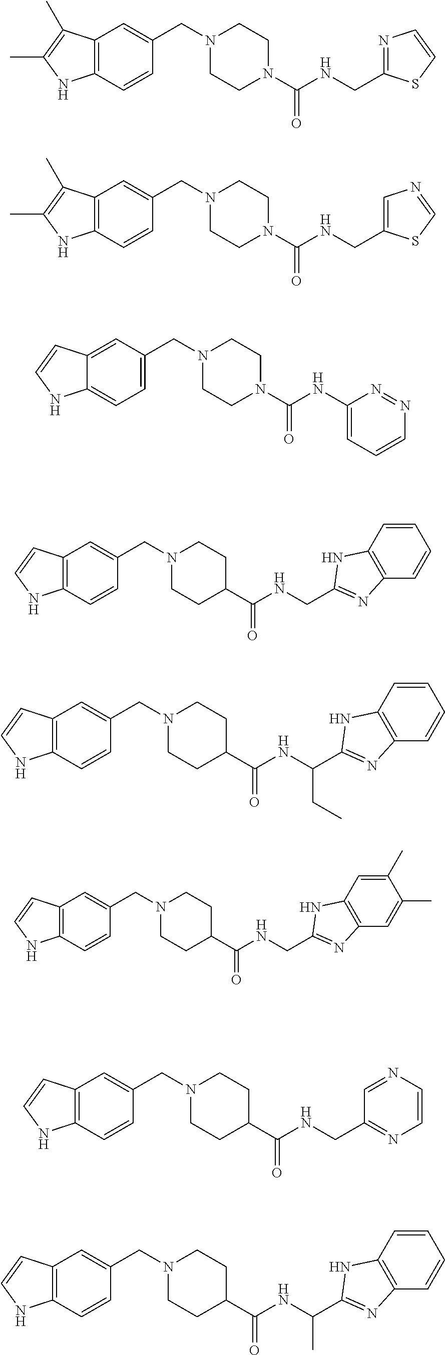

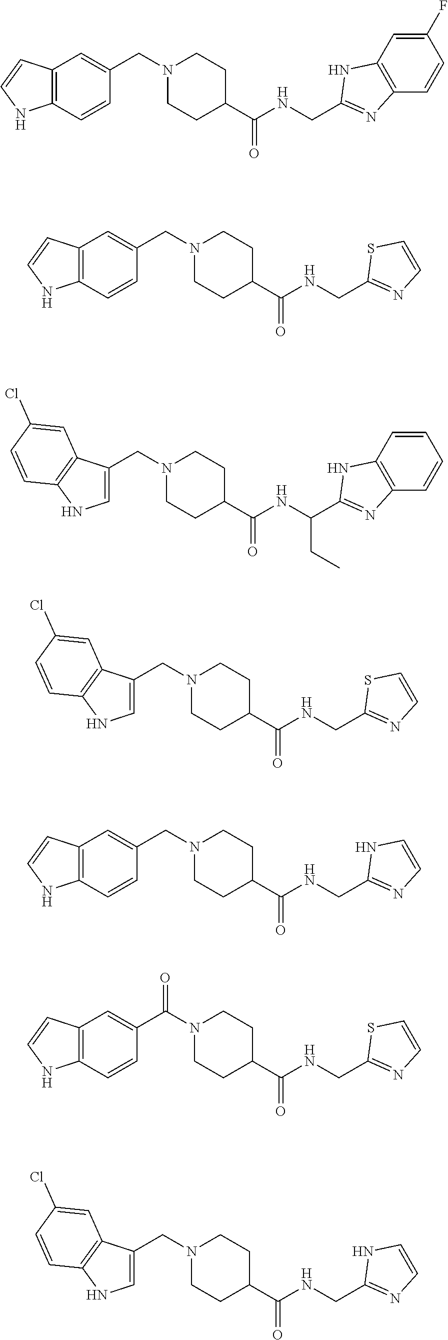

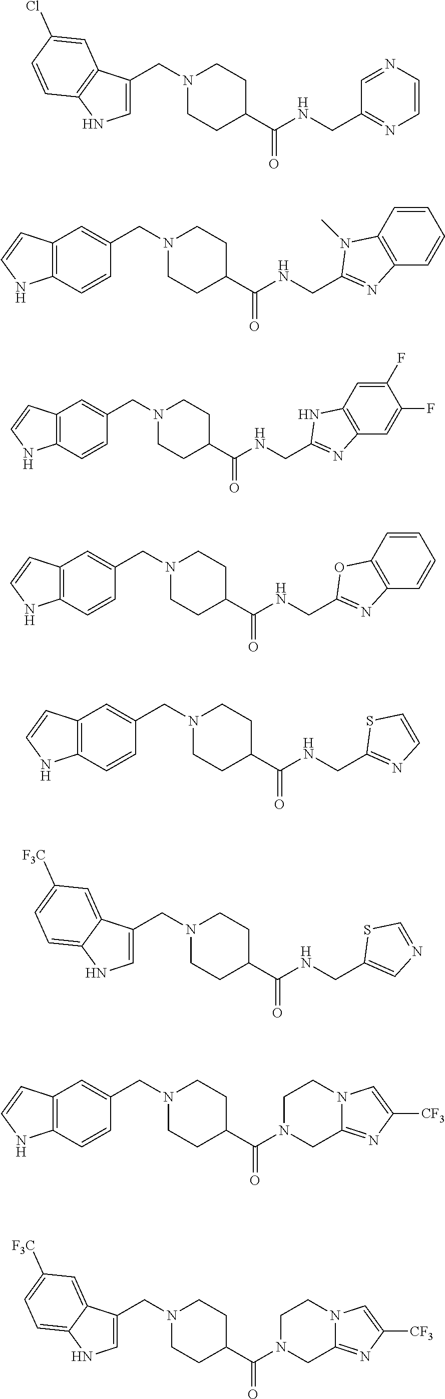

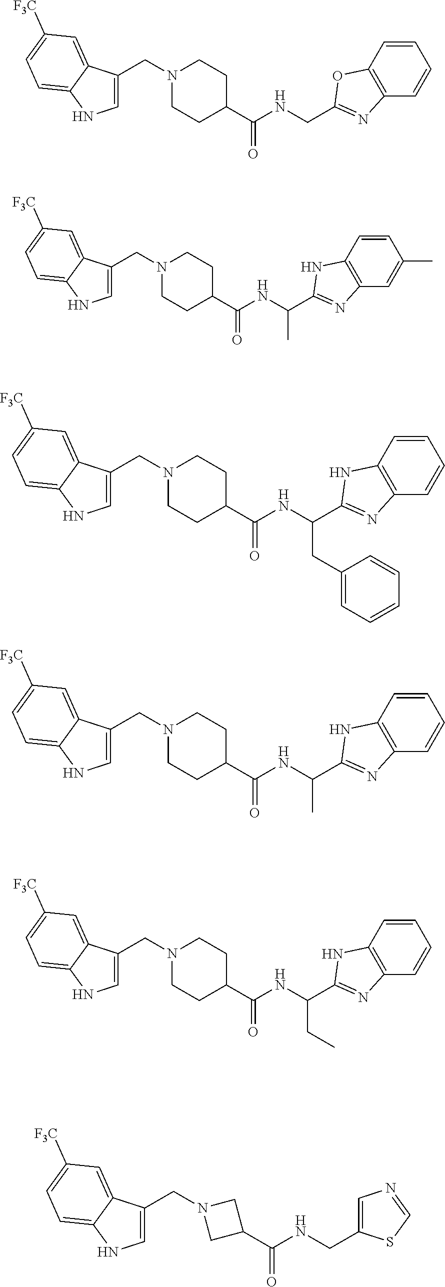 [SMILES: O=C(NCc1nc2ccccc2o1)C1CCN(Cc2c[nH]c3ccc(C(F)(F)F)cc23)CC1 ; Cc1ccc2[nH]c(C(C)NC(=O)C3CCN(Cc4c[nH]c5ccc(C(F)(F)F)cc45)CC3)nc2c1 ; O=C(NC(Cc1ccccc1)c1nc2ccccc2[nH]1)C1CCN(Cc2c[nH]c3ccc(C(F)(F)F)cc23)CC1 ; CC(NC(=O)C1CCN(Cc2c[nH]c3ccc(C(F)(F)F)cc23)CC1)c1nc2ccccc2[nH]1 ; CCC(NC(=O)C1CCN(Cc2c[nH]c3ccc(C(F)(F)F)cc23)CC1)c1nc2ccccc2[nH]1 ; O=C(NCc1cncs1)C1CN(Cc2c[nH]c3ccc(C(F)(F)F)cc23)C1]

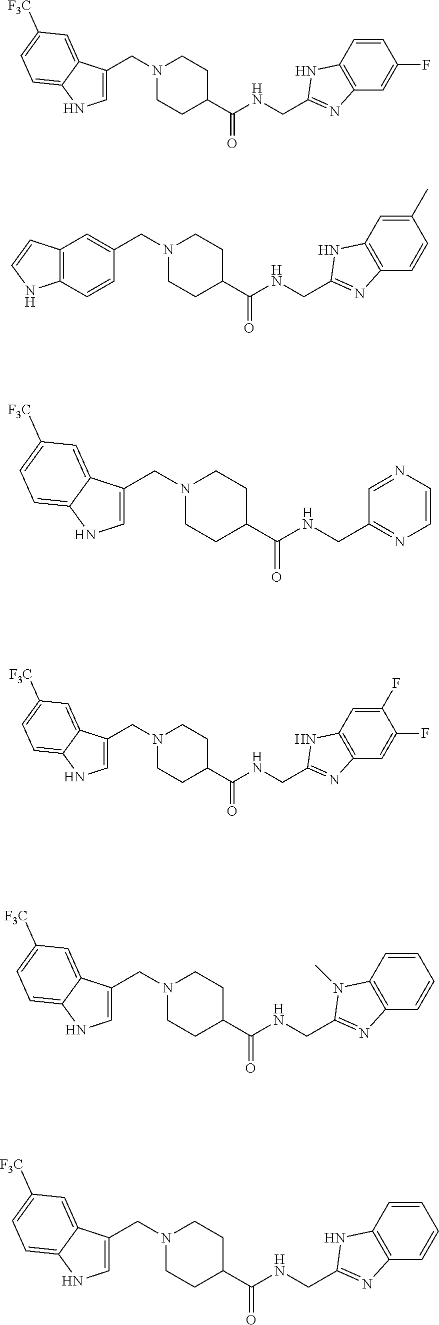 [SMILES: O=C(NCc1nc2cc(F)ccc2[nH]1)C1CCN(Cc2c[nH]c3ccc(C(F)(F)F)cc23)CC1 ; Cc1ccc2nc(CNC(=O)C3CCN(Cc4ccc5[nH]ccc5c4)CC3)[nH]c2c1 ; O=C(NCc1cnccn1)C1CCN(Cc2c[nH]c3ccc(C(F)(F)F)cc23)CC1 ; O=C(NCc1nc2cc(F)c(F)cc2[nH]1)C1CCN(Cc2c[nH]c3ccc(C(F)(F)F)cc23)CC1 ; Cn1c(CNC(=O)C2CCN(Cc3c[nH]c4ccc(C(F)(F)F)cc34)CC2)nc2ccccc21 ; O=C(NCc1nc2ccccc2[nH]1)C1CCN(Cc2c[nH]c3ccc(C(F)(F)F)cc23)CC1]

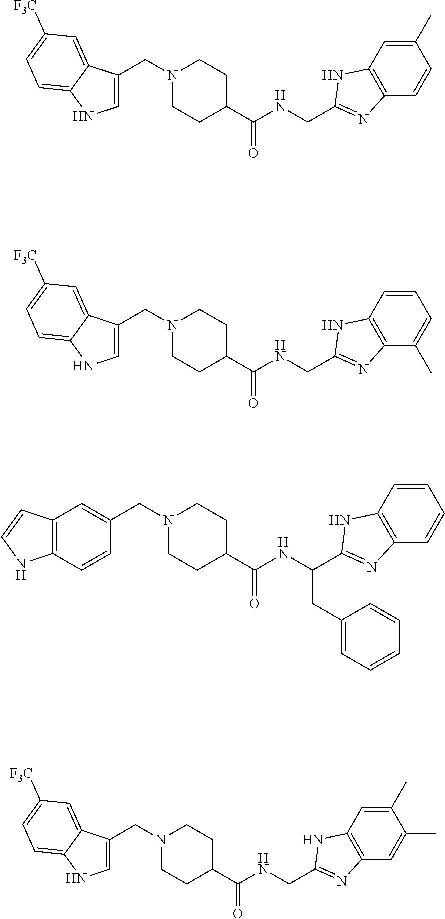 [SMILES: Cc1ccc2nc(CNC(=O)C3CCN(Cc4c[nH]c5ccc(C(F)(F)F)cc45)CC3)[nH]c2c1 ; Cc1cccc2[nH]c(CNC(=O)C3CCN(Cc4c[nH]c5ccc(C(F)(F)F)cc45)CC3)nc12 ; O=C(NC(Cc1ccccc1)c1nc2ccccc2[nH]1)C1CCN(Cc2ccc3[nH]ccc3c2)CC1 ; Cc1cc2nc(CNC(=O)C3CCN(Cc4c[nH]c5ccc(C(F)(F)F)cc45)CC3)[nH]c2cc1C]

The compound of Formula 1 according to the present invention may be prepared by methods representatively shown in Schemes 1 to 8:

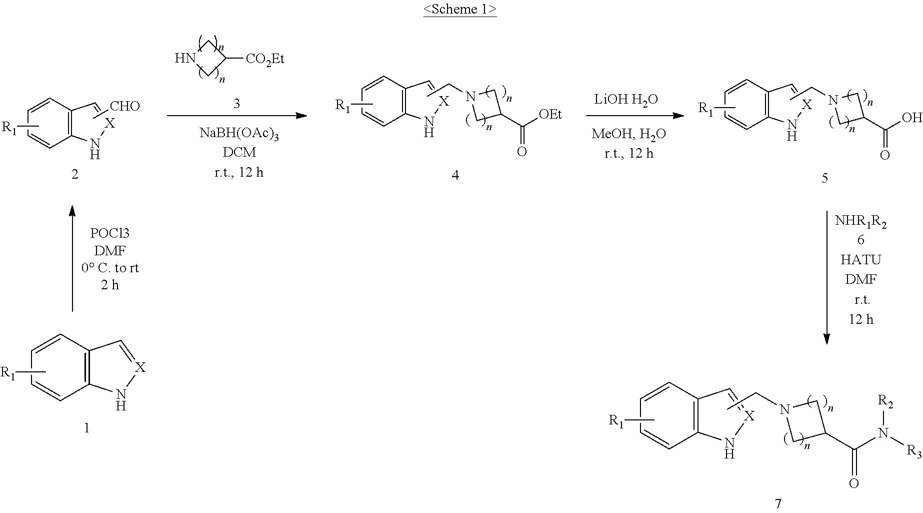

A substituted or unsubstituted indole-aldehyde starting material (2, where X is CH or N), which is prepared by treating an indol starting material (1, where X=CH) with POCl₃ or is commercially available, was subjected to a reductive amination reaction with various amines (3) (where n's are each independently 1 or 2), to prepare an ester intermediate compound (4). Various acid intermediate compounds (5), which are prepared by treating the ester intermediate compound (4) with LiOH or is directly commercially available, were subjected to a coupling reaction with various amines (6), to prepare a final compound (7).

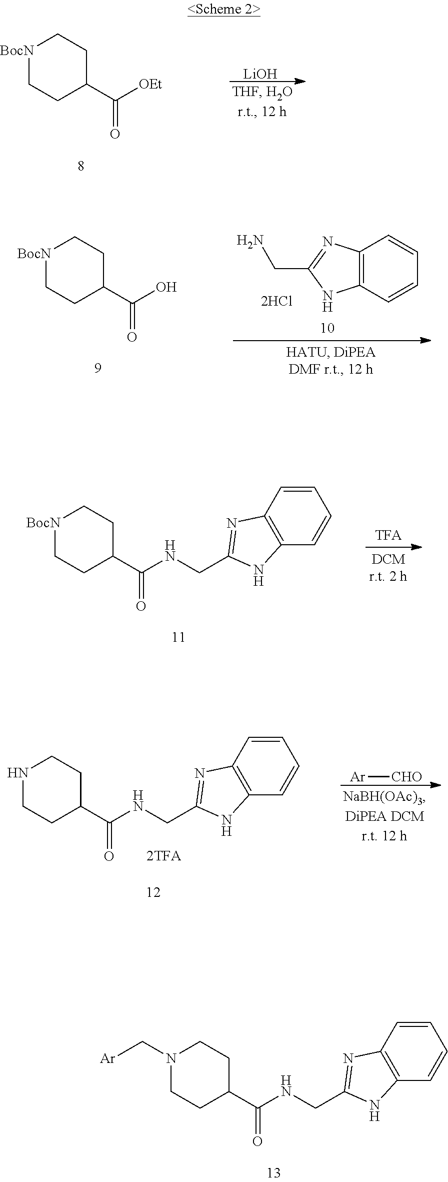

Unlike Scheme 1, it was also possible to synthesize a final target compound (13) of the present invention as follows. An acid intermediate compound (9), obtained by treating a piperidine-ester starting material (8) with LiOH, was subjected to a standard amide coupling reaction with amine (10) containing a benzimidazole heterocycle, to synthesize an amide intermediate compound (11). The amide intermediate compound (11) was treated with TFA, to prepare an amine intermediate compound (12) with an amine protecting group removed. Then, the amine intermediate compound (12) was reacted with various aldehyde compounds, to synthesize the final target compound (13).

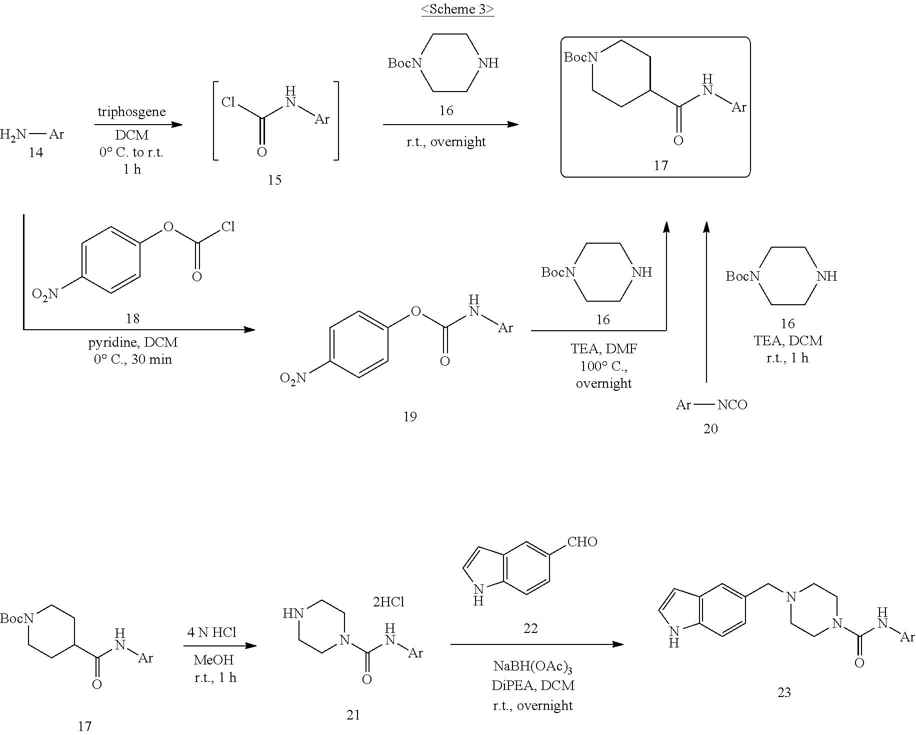

On the other hand, in order to prepare a compound such as a final compound (23) containing piperazine-urea according to the present invention, it was possible to use the following method. A Boc-protected piperazine urea intermediate compound (17) was prepared, in general, through a method in which amine (14) is reacted with triphosgene and then reacted with piperazine protected with Boc at one side, or a method in which amine (14) is reacted with 4-nitrophenyl carbamoyl chloride and then reacted with piperazine protected with Boc at one side. The intermediate compound was treated with HC1 to obtain an amine intermediate compound (18). Then, the amine intermediate compound was subjected to a reductive amination reaction with an aldehyde intermediate compound (21), to prepare the final compound (23).

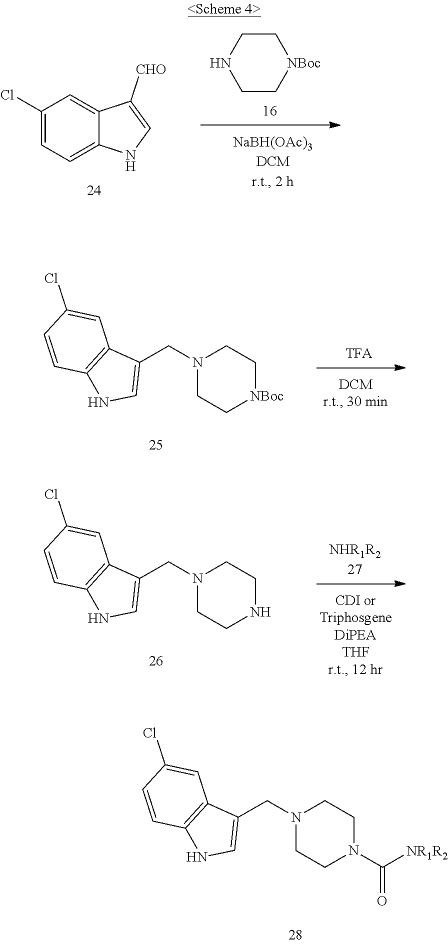

In addition, for synthesis of a derivative such as a final compound (28) containing piperazine urea according to the present invention, it was also possible to use a method as in Scheme 4. A chloro-indole aldehyde intermediate compound (24) as a starting material was subjected to a reductive amination reaction with Boc-piperazine (16), to prepare an indole-substituted piperazine intermediate compound (25). The resulting compound was treated with TFA to prepare an amine intermediate compound (26). Then, the amine intermediate compound was subjected to urea synthesis using CDI or triphosgene/DIPEA with various amines (27), to synthesize various final compounds (28) of the present invention.

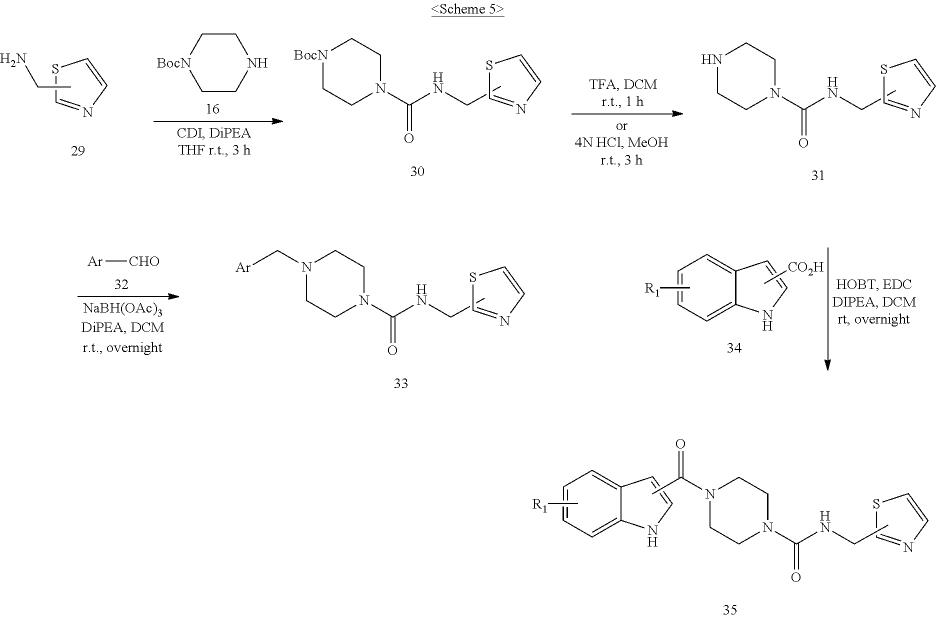

For synthesis of a final derivative compound (33) containing thiazole, it was also possible to synthesize the same using thiazole-methyl amine (29) as a starting material. A urea intermediate compound (30) was prepared using the urea synthesis described in Scheme 4 and deprotected using TFA. Then, the resulting product was subjected to a reductive amination reaction with various aldehyde intermediate compounds (32), which made it possible to synthesize the final compound (33) of the present invention; or the resulting product was subjected to an amide coupling reaction with various acid intermediate compounds (34), which also made it possible to prepare a final compound (35) of the present invention.

The compound of Formula 1 according to the present invention may be prepared in the form of a pharmaceutically acceptable salt obtained by addition of an inorganic or organic acid. Here, a preferred salt thereof may include a salt derived from hydrochloric acid, hydrobromic acid, sulfuric acid, phosphoric acid, nitric acid, acetic acid, glycolic acid, lactic acid, pyruvic acid, malonic acid, succinic acid, glutaric acid, fumaric acid, malic acid, mandelic acid, tartaric acid, citric acid, ascorbic acid, palmitic acid, maleic acid, hydroxymaleic acid, benzoic acid, hydroxybenzoic acid, phenylacetic acid, cinnamic acid, salicylic acid, methanesulfonic acid, benzenesulfonic acid, toluenesulfonic acid, or the like.

Specifically, the pharmaceutically acceptable salt according to the present invention may be prepared by dissolving the compound of Formula 1 in an organic solvent, for example, acetone, methanol, ethanol, acetonitrile, or the like, and filtering the precipitated crystal by addition of an organic acid or an inorganic acid. Alternatively, the pharmaceutically acceptable salt according to the present invention may be prepared by subjecting solvent or excess acid in a reaction mixture, to which acid has been added, to reduced pressure evaporation so that a residue is dried, or by filtering a salt precipitated by addition of another organic solvent.

The compound of Formula 1 according to the present invention or a pharmaceutically acceptable salt thereof may be in the form of a hydrate or solvate, and such compounds are also included in the present invention.

The compound of Formula 1 according to the present invention or a pharmaceutically acceptable salt thereof can effectively inhibit protein kinases. In an embodiment, the compound of the present invention can be effectively used to prevent or treat a disease mediated by simultaneous expression of p34 and NEDD4-1. That is, as the compound of the present invention binds to a portion of p34 protein and binds to a portion of NEDD4-1 protein, it is possible to inhibit binding between the p34 protein and the NEDD4-1 protein. Specifically, the disease may be selected from metastatic tumor growth, but is not limited thereto.

In a specific embodiment of the present invention, the compound of Formula 1 or a pharmaceutically acceptable salt thereof can be effectively used to prevent or treat cancer or tumor, and furthermore, can also be effectively used to inhibit metastasis of cancer cells. Here, the cancer may be selected from, but is not limited to, the group consisting of liver cancer, hepatocellular carcinoma, thyroid cancer, colorectal cancer, testicular cancer, bone cancer, oral cancer, basal cell carcinoma, ovarian cancer, brain tumor, gallbladder carcinoma, biliary tract cancer, head and neck cancer, colorectal cancer, vesical carcinoma, tongue cancer, esophageal cancer, glioma, glioblastoma, renal cancer, malignant melanoma, gastric cancer, breast cancer, sarcoma, pharynx carcinoma, uterine cancer, cervical cancer, prostate cancer, rectal cancer, pancreatic cancer, lung cancer, colorectal cancer, colon cancer, and other solid cancers.

In a specific embodiment of the present invention, the compound of Formula 1 or a pharmaceutically acceptable salt thereof can be effectively used to prevent or treat carcinoma mediated by simultaneous expression of p34 and NEDD4-1. Here, the carcinoma may be selected from, but is not limited to, the group consisting of carcinomas of lung, liver, biliary tract, gastrointestinal tract, head and neck, pancreas, prostate, cervix, breast, colorectum, and colon.

The compound of Formula 1 according to the present invention, a pharmaceutically acceptable salt thereof, or the like may be administered to a subject in order to prevent or treat the disease. Here, the dosage may vary depending on the subject to be treated, the severity of disease or condition, the rate of administration, and the judgment of the prescribing physician. Ordinarily, the compound of Formula 1 may be administered, via an oral or parenteral route, to a human as an active ingredient in an amount of 0.1 to 2,000 mg/day, preferably 1 to 1,000 mg/day, on a 70-kg body weight basis, 1 to 4 times daily or on an on/off schedule. In some cases, fewer dosages than the above-mentioned ranges may be more suitable, and more dosages may be used without causing deleterious side effects. In a case where more dosages are used, such dosages are divided into several smaller dosages over the day.

The pharmaceutical composition according to the present invention may be formulated according to a conventional method, and may be prepared in various oral dosage forms such as tablets, pills, powders, capsules, syrups, emulsions, and microemulsions, or in parenteral dosage forms such as for intramuscular, intravenous, or subcutaneous administration.

In a case where the pharmaceutical composition according to the present invention is prepared in the form of an oral formulation, examples of a carrier used may include cellulose, calcium silicate, corn starch, lactose, sucrose, dextrose, calcium phosphate, stearic acid, magnesium stearate, calcium stearate, gelatin, talc, a surfactant, a suspending agent, an emulsifying agent, a diluent, and the like. In a case where the pharmaceutical composition according to the present invention is prepared in the form of an injection, for the carrier, water, saline, aqueous glucose solution, aqueous similar sugar solution, alcohol, glycol, ether (for example, Polyethylene Glycol 400), oil, fatty acid, fatty acid ester, glyceride, a surfactant, a suspending agent, an emulsifier, and the like may be used.

Hereinafter, the present invention will be described in detail by way of examples. However, the following examples are merely given to illustrate the present invention, and the scope of the present invention is not limited by these examples.

### EXAMPLES

### Example 1: N-((1H-benzo[d]imidazol-2-yl)methyl)-1-((1H-indol-5-yl) methyl)piperidine-4-carboxamide

### Step 1) Ethyl 1-((1H-indol-5-yl)methyl)piperidine-4-carboxylate

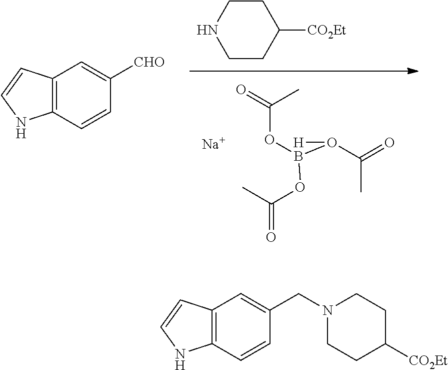

A solution of 1H-indole-5-carbaldehyde (0.200 g, 1.378 mmol) and ethyl piperidine-4-carboxylate (0.238 g, 1.516 mmol) in DCM (6.89 ml) was stirred at room temperature for 1 hour. Sodium triacetoxyhydroborate (0.438 g, 2.067 mmol) was added dropwise thereto at once. Then, the reaction mixture was stirred at room temperature for 12 hours and quenched with saturated aqueous NaHCO₃ solution. The mixture was extracted with DCM twice. The combined organic layers were dried over Na₂SO₄, filtered, and concentrated in vacuo. The residue was purified by column chromatography on silica gel (SiO₂) (Hex:EtOAc=1:1 to 1:2 to EtOAc only), to obtain ethyl 1-((1H-indol-5-yl)methyl) piperidine-4-carboxylate (0.364 g, 1.271 mmol, 92% yield) as clear oil.

MS (ESI) m/z 286 (M⁺+1)

### Step 2) 1-((1H-indol-5-yl)methyl)piperidine-4-carboxylic acid

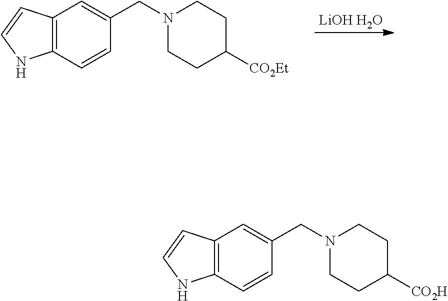 [SMILES: CCOC(=O)C1CCN(Cc2ccc3[nH]ccc3c2)CC1 ; "LiOH H2O" ; O=C(O)C1CCN(Cc2ccc3[nH]ccc3c2)CC1]

A mixture of ethyl 1-((1H-indol-5-yl)methyl)piperidine-4-carboxylate (0.364 g, 1.271 mmol) and lithium hydroxide hydrate (0.107 g, 2.54 mmol) in a mixed solvent of MeOH (4.77 ml) and water (1.589 ml) was stirred overnight at room temperature. Neutralization with 2N HCl aqueous solution was performed, and then the mixture was concentrated in vacuo. A process in which the residue is diluted with toluene and concentrated in vacuo was performed twice, to obtain 1-((1H-indol-5-yl)methyl)piperidine-4-carboxylic acid as an orange solid containing LiCl, which was used in the next step without further purification.

MS (ESI) m/z 258 (M⁺+1)

### Step 3) N-((1H-benzo[d]imidazol-2-yl)methyl)-1-((1H-indol-5-yl)methyl)piperidine-4-carboxamide

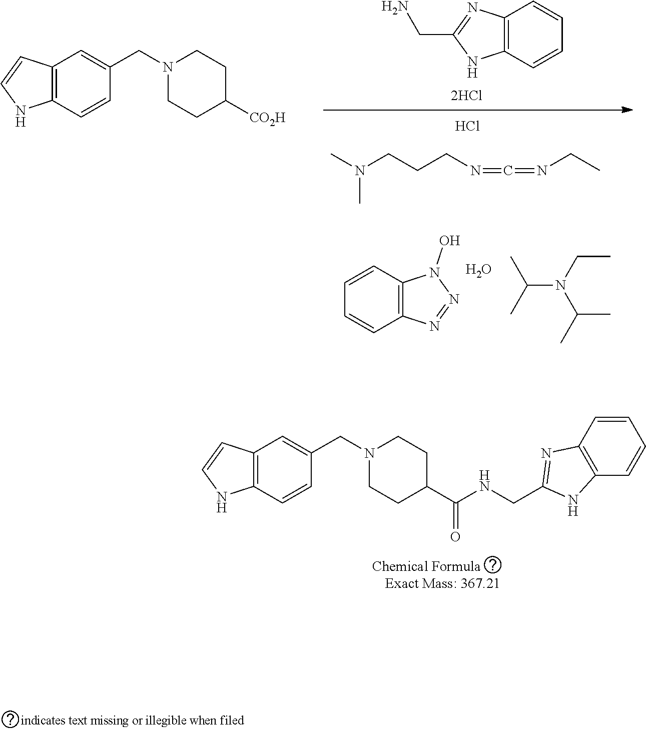

To a mixture of 1-((1H-indol-5-yl)methyl)piperidine-4-carboxylic acid (0.164 g, 0.635 mmol) in DMF (3.17 ml) was added at room temperature N1-((ethylimino)methylene)-N3,N3-dimethylpropane-1,3 -diamine hydrochloride (0.183 g, 0.952 mmol), (1H-benzo [d]imidazol-2-yl)methylamine (0.762 mmol, 1.2 eq), and triethylamine (0.762 mmol, 1.2 eq). The mixture was stirred at room temperature for 10 minutes. 1H-benzo[d][1,2,3]triazole-1-ol hydrate (0.146 g, 0.952 mmol) was added thereto, and the resulting reaction mixture was stirred overnight at room temperature. The reaction mixture was partitioned between DCM and water. The separated water layer was extracted with DCM. The combined organic layers were washed with water and brine, dried over Na₂SO₄, filtered, and concentrated in vacuo. The residue was purified by column chromatography on silica gel (SiO₂) (EtOAc only to EtOAc:MeOH=10:1 to 5:1), to obtain N-((1H-benzo[d]imidazol-2-yl)methyl)-1-((1H-indol-5-yl)methyl)piperidine-4-carboxamide (0.120 g, 0.310 mmol, 48.8% yield) as an ivory solid.

¹H NMR (DMSO-d₆) δ 1.06 (s, 1H), 1.48 (d, 3H), 1.66 (s, 4H), 2.15 (s, 2H), 2.32 (s, 1H), 2.68 (s, 5H), 2.72 (s, 2H), 2.88 (s, 4H), 3.47 (s, 2H), 5.09-5.16 (m, 1H), 6.36 (s, 1H), 7.13 (s, 3H), 7.30 (d, 5H), 7.95 (s, 1H), 8.33 (d, 1H), 11.00 (s, 1H), 12.13 (s, 1H).

Compounds of Examples 2 to 61 were synthesized in the same manner as in Example 1, except that appropriate aldehydes are used which correspond to 1H-indole-5-carbaldehyde used in step 1 of Example 1, and adequate amines are used in place of (1H-benzo[d]imidazol-2-yl)methylamine in step 3 of Example 1. However, for the compound of Example 36, in addition to the above exception, such a compound was synthesized in the same manner as in Example 1, except that methyl 1-((1H-indol-3-yl)methyl)azetidine-3-carboxylate is used in place of ethyl piperidine-4-carboxylate used in step 1 of Example 1.

The compounds of Examples 2 to 61 synthesized in the same manner as described above are shown in Table 1.

TABLE 1

| Example | Chemical structure | NMR spectrum data |
|---|---|---|
| 2 | 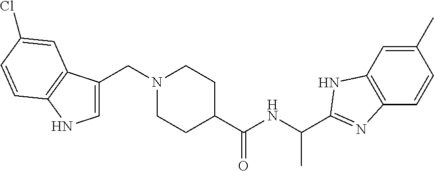 [SMILES: Cc1ccc2nc(C(C)NC(=O)C3CCN(Cc4c[nH]c5ccc(Cl)cc45)CC3)[nH]c2c1] | ¹H NMR (DMSO-d₆) δ 0.77-0.82(m, 1H), 1.19(s, 1H), 2.12 (t, 1H), 2.63(t, 1H), 2.71 (s, 2H), 2.95(s, 1H), 3.12(s, 4H), 5.04-5.12(m, 1H), 7.00(d, 2H), 7.02(d, 4H), 7.59(s, 1H), 7.91(s, 1H), 8.75(d, 1H), 11.14(s, 1H) |
| 3 | 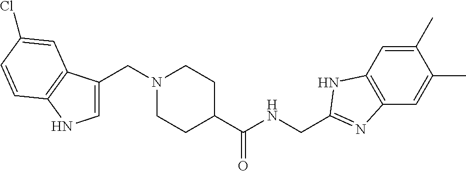 [SMILES: Cc1cc2nc(CNC(=O)C3CCN(Cc4c[nH]c5ccc(Cl)cc45)CC3)[nH]c2cc1C] | ¹H NMR (DMSO-d₆) δ 1.19(s, 1H), 1.83(t, 2H), 2.46(t, 3H), 2.92(s, 1H), 3.34(s, 2H), 3.52(s, 3H), 4.39(d, 2H), 7.02(d, 1H), 7.24(s, 1H), 7.25(s, 1H), 7.32(d, 1H), 7.59(s, 1H), 8.74(t, 1H), 11.16(d, 1H) |
| 4 | 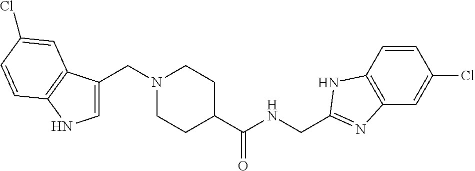 [SMILES: O=C(NCc1nc2cc(Cl)ccc2[nH]1)C1CCN(Cc2c[nH]c3ccc(Cl)cc23)CC1] | ¹H NMR (DMSO-d₆) δ 0.94(d, 1H), 1.19(t, 2H), 1.52-1.59(m, 2H), 1.86(t, 2H), 1.98(s, 1H), 2.86(s, 2H), 2.72(s, 2H), 4.01-4.03(m, 1H), 4.45(d, 2H), 7.12(d, 1H), 7.15(d, 1H), 7.29(s, 1H), 7.36(d, 1H), 7.45-7.51(m, 1H), 7.63(s, 1H), 8.99(s, 1H), 11.19(s, 1H) |
| 5 | 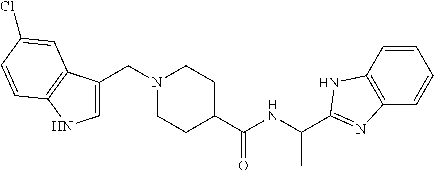 [SMILES: CC(NC(=O)C1CCN(Cc2c[nH]c3ccc(Cl)cc23)CC1)c1nc2ccccc2[nH]1] | ¹H NMR (DMSO-d₆) δ 0.88(t, 1H), 1.25(s, 1H), 1.30(s, 3H), 1.46(d, 4H), 1.87(s, 5H), 2.31(s, 1H), 2.88(d, 2H), 5.09-5.17(m, 1H), 7.06(d, 1H), 7.10- 7.14(m, 1H), 7.29(d, 1H), 7.36(s, 1H), 7.47-7.49(m, 1H), 7.63(d, 1H), 8.40(d, 1H), 11.15(s, 1H) |
| 6 | 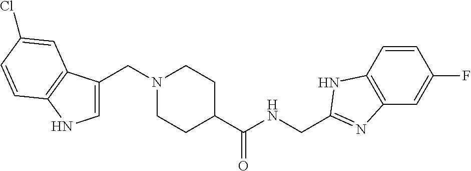 [SMILES: O=C(NCc1nc2cc(F)ccc2[nH]1)C1CCN(Cc2c[nH]c3ccc(Cl)cc23)CC1] | ¹H NMR (DMSO-d₆) δ 0.84-0.89(m, 1H), 1.16-1.23(m, 1H), 1.48-1.62(m, 2H), 1.73(d, 2H), 1.90(t, 3H), 2.15- 2.21(m, 1H), 2.92(d, 2H), 3.17(s, 1H), 3.59(s, 2H), 4.45(s, 2H), 6.98(t, 1H), 7.08(d, 1H), 7.30(s, 1H), 7.37 (d, 1H), 7.47-7.48 (m, 1H), 7.65(s, 1H), 8.46(s, 1H), 11.17(s, 1H) |
| 7 | 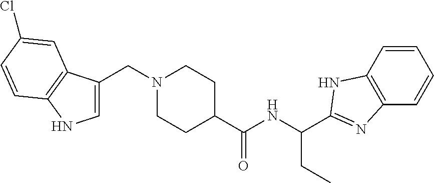 [SMILES: CCC(NC(=O)C1CCN(Cc2c[nH]c3ccc(Cl)cc23)CC1)c1nc2ccccc2[nH]1] | ¹H NMR (DMSO-d₆) δ 0.83-0.96(m, 3H), 1.17(t, 1H), 2.33(s, 2H), 2.88(s, 2H), 3.57(d, 2H), 4.03(d, 1H), 4.93- 4.99(m, 1H), 7.12(m, 1H), 7.29(s, 1H), 7.36(d, 1H), 7.52(d, 1H), 7.64(s, 1H), 8.23(d, 1H), 11.11(s, 1H), 12.14(s, 1H) |
| 8 | 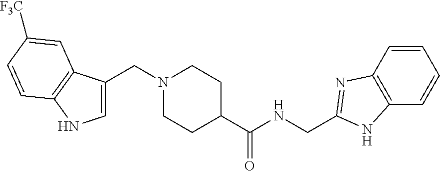 [SMILES: O=C(NCc1nc2ccccc2[nH]1)C1CCN(Cc2c[nH]c3ccc(C(F)(F)F)cc23)CC1] | ¹H NMR (MeOH-d₄) δ 1.77-1.91(m, 5H), 2.16(t, 2H), 2.26-2.31(m, 1H), 3.07(d, 2H), 3.79(s, 2H), 4.56(s, 2H), 2.17-7.19(m, 2H), 7.35(d, 2H), 7.49(d, 3H), 7.97(s, 1H) |
| 9 | 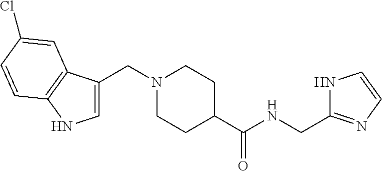 [SMILES: O=C(NCc1ncc[nH]1)C1CCN(Cc2c[nH]c3ccc(Cl)cc23)CC1] | ¹H NMR (DMSO-d₆) δ 0.84-0.87(m, 1H), 1.54-1.62(m, 1H), 1.84(s, 1H), 2.85(d, 2H), 3.16(s, 1H), 3.55(s, 2H), 4.23(s, 2H), 6.86(s, 1H), 7.05(d, 1H), 7.27(s, 1H), 7.36(d, 1H), 7.62(s, 1H), 8.48(s, 1H), 11.26(s, 1H) |
| 10 | 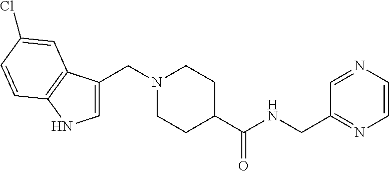 [SMILES: O=C(NCc1cnccn1)C1CCN(Cc2c[nH]c3ccc(Cl)cc23)CC1] | ¹H NMR (DMSO-d₆) δ 0.85(s, 1H), 1.22(s, 1H), 2.12-2.24(m, 1H), 2.61(s, 1H), 2.89(d, 2H), 3.16(s, 1H), 3.36(s, 1H), 3.56(s, 2H), 4.38(d, 3H), 7.05(d, 1H), 7.28(s, 1H), 7.36(s, 1H), 7.63(d, 1H), 8.50-8.56(m, 3H), 11.27(s, 1H) |
| 11 | 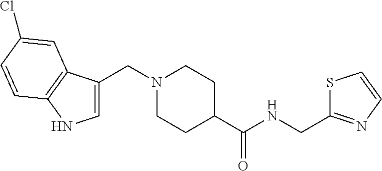 [SMILES: O=C(NCc1nccs1)C1CCN(Cc2c[nH]c3ccc(Cl)cc23)CC1] | ¹H NMR (DMSO-d₆) δ 1.06(s, 1H), 1.23(s, 1H), 1.51-1.60(m, 3H), 1.85(s, 6H), 1.99(m, 1H), 2.06(s, 1H), 2.87(d, 3H), 4.44(d, 2H), 7.06(d, 1H), 7.33(d, 1H), 7.62(d, 1H), 7.71(d, 1H), 8.43(t, 1H), 8.93(s, 1H), 11.12(s, 1H) |
| 12 | 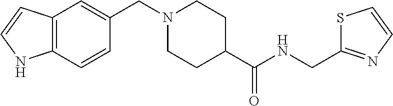 [SMILES: O=C(NCc1nccs1)C1CCN(Cc2ccc3[nH]ccc3c2)CC1] | ¹H NMR (DMSO-d₆) δ 1.53-1.60(m, 4H), 1.87(t, 2H), 2.05-2.11(m, 1H), 2.88(d, 2H), 3.47(s, 2H), 4.44(d, 2H), 6.36(s, 1H), 7.02(d, 1H), 7.28-7.32(m, 2H), 7.39(s, 1H), 7.71(s, 1H), 8.44(t, 1H), 8.93(s, 1H), 11.00(s, 1H) |
| 13 | 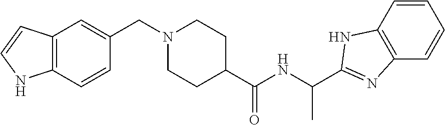 [SMILES: CC(NC(=O)C1CCN(Cc2ccc3[nH]ccc3c2)CC1)c1nc2ccccc2[nH]1] | ¹H NMR (DMSO-d₆) δ 1.06(s, 1H), 1.48(d, 3H), 1.66(s, 4H), 1.89(s, 3H), 2.15-2.18(m, 1H), 2.32(s, 1H), 2.68(s, 5H), 2.72(s, 2H), 2.88(s, 4H), 3.47(s, 2H), 5.09-5.16(m, 1H), 6.36(s, 1H), 7.13(s, 3H), 7.30(d, 5H), 7.95(s, 1H), 8.33(d, 1H), 11.00(s, 1H), 12.13(s, 1H) |
| 14 | 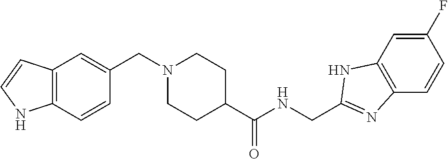 [SMILES: O=C(NCc1nc2ccc(F)cc2[nH]1)C1CCN(Cc2ccc3[nH]ccc3c2)CC1] | ¹H NMR (DMSO-d₆) δ 1.53-1.62(m, 2H), 1.71(s, 2H), 1.88(t, 2H), 2.15- 2.21(m, 1H), 2.32(s, 1H), 2.68(s, 2H), 2.88(s, 3H), 3.16(s, 1H), 3.47(s, 2H), 4.40(d, 2H), 6.36(s, 1H), 6.96-7.03(m, 2H), 7.28-7.32(m, 3H), 7.40(s, 2H), 7.95(s, 1H), 8.43(t, 1H) |
| 15 | 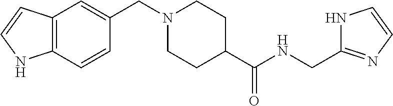 [SMILES: O=C(NCc1ncc[nH]1)C1CCN(Cc2ccc3[nH]ccc3c2)CC1] | ¹H NMR (DMSO-d₆) δ 0.86(s, 1H), 1.23(s, 1H), 2.83(d, 2H), 3.16(s, 1H), 3.46(s, 2H), 6.35(s, 1H), 6.85(s, 2H), 7.02(d, 1H), 7.31(s, 2H), 7.39(s, 1H), 8.70(t, 1H) |
| 16 | 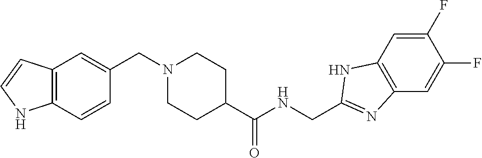 [SMILES: O=C(NCc1nc2cc(F)c(F)cc2[nH]1)C1CCN(Cc2ccc3[nH]ccc3c2)CC1] | ¹H NMR (DMSO-d₆) δ 3.47(s, 2H), 3.58(s, 1H), 4.43(s, 1H), 6.36(s, 1H), 7.03(d, 1H), 7.12(d, 2H), 7.49(s, 1H), 7.49-7.53(m, 2H) |
| 17 | 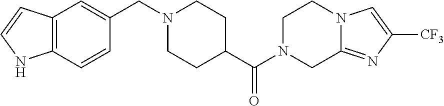 [SMILES: O=C(C1CCN(Cc2ccc3[nH]ccc3c2)CC1)N1CCn2cc(C(F)(F)F)nc2C1] | ¹H NMR (DMSO-d₆) δ 1.54(s, 4H), 1.92-1.97(m, 2H), 2.68(s, 1H), 2.84(s, 2H), 3.12(d, 1H), 3.46(s, 2H), 4.07(s, 4H), 4.59(s, 1H), 3.5(s, 2H), 4.43(s, 2H), 6.32(s, 1H), 6.99(d, 1H), 7.26- 7.28(m, 2H), 7.32(s, 1H), 7.4(s, 1H), 7.71(s, 1H), 8.42-8.48(t, 1H), 8.92(s, |
| | | 1H) |
| 18 | 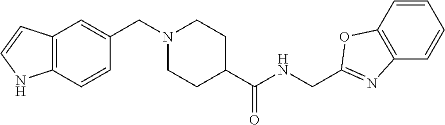 [SMILES: O=C(NCc1nc2ccccc2o1)C1CCN(Cc2ccc3[nH]ccc3c2)CC1] | ¹H NMR (DMSO-d₆) δ 1.62(s, 4H), 1.85(t, 2H), 2.12-2.15(m, 1H), 2.68(s, 1H), 2.81(d, 2H), 3.12(d, 0.51H), 3.43(s, 2H), 4.49(d, 2H), 6.31(s, 1H), 6.99(d, 1H), 7.25(m, 5H), 7.64(d, 2H), 8.53(t, 1H), 10.96(s, 1H) |
| 19 | 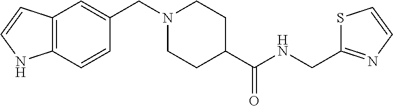 [SMILES: O=C(NCc1nccs1)C1CCN(Cc2ccc3[nH]ccc3c2)CC1] | ¹H NMR (DMSO-d₆) δ 1.50-1.64(m, 4H), 1.94(s, 2H), 2.12(t, 1H), 2.84(d, 2H), 3.44(s, 2H), 4.47(d, 2H), 6.32(s, 1H), 6.99(d, 1H), 7.26(s, 3H), 7.54(d, 2H), 8.14(t, 1H) |
| 20 | 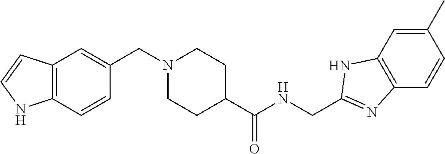 [SMILES: Cc1ccc2nc(CNC(=O)C3CCN(Cc4ccc5[nH]ccc5c4)CC3)[nH]c2c1] | ¹H NMR (DMSO-d₆) δ 1.48-1.58(m, 2H), 1.66(d, 2H), 1.84(t, 2H), 2.14(t, 1H), 2.33(s, 3H), 2.82(d, 2H), 3.12(s, 1H), 3.29(s, 3H), 3.43(s, 2H), 4.07(d, 1H), 4.38(d, 2H), 6.32(s, 1H), 6.91(d, 1H), 6.99(d, 1H), 7.36(s, 5H), 8.38(s, 1H) |
| 21 | 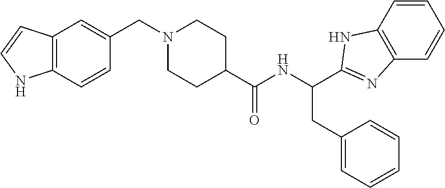 [SMILES: O=C(NC(Cc1ccccc1)c1nc2ccccc2[nH]1)C1CCN(Cc2ccc3[nH]ccc3c2)CC1] | ¹H NMR (DMSO-d₆) δ 1.18(t, 2H), 1.35-1.36(m, 1H), 1.47-1.54(m, 2H), 1.81-1.84(m, 2H), 1.99(s, 2H), 2.11- 2.12(m, 1H), 2.33(t, 1H), 2.67-2.78(m, 2H), 3.07-3.11(m, 1H), 3.34-3.45(m, 11H), 4.01-4.06(m, 1H), 5.28-5.34(m, 1H), 6.37(s, 1H), 7.14-7.39(m, 14H), 8.36(d, 1H), 8.56(d, 1H), 11.00(s, 1H), 12.22(s, 1H) |
| 22 | 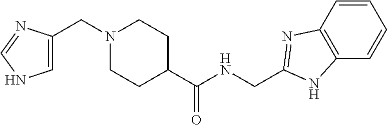 [SMILES: O=C(NCc1nc2ccccc2[nH]1)C1CCN(Cc2c[nH]cn2)CC1] | ¹H NMR (DMSO-d₆) δ 1.58(t, 3H), 1.72(d, 2H), 1.88(t, 2H), 2.16(t, 1H), 2.86(d, 2H), 3.37(d, 3H), 4.46(d, 2H), 6.83(s, 1H), 7.13(s, 2H), 7.52(d, 3H), 8.40(s, 1H), 11.89(s, 1H), 12.14(s, 1H) |
| 23 | 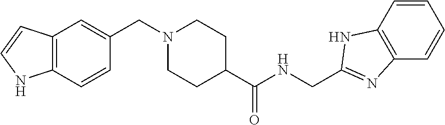 [SMILES: O=C(NCc1nc2ccccc2[nH]1)C1CCN(Cc2ccc3[nH]ccc3c2)CC1] | ¹H NMR (DMSO-d₆) δ 1.57(d, 4H), 1.69(s, 3H), 1.90(s, 1H), 2.49(s, 1H), 2.68(d, 2H), 2.87(d, 2H), 3.48(s, 2H), 4.45(d, 2H), 6.36(s, 1H), 7.32(d, 3H), 7.40(s, 5H), 7.95(s, 1H), 8.41(t, 1H), 11.01(s, 1H), 12.13(s, 1H) |
| 24 | 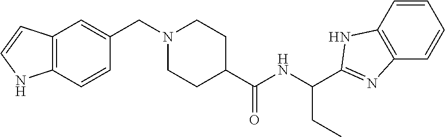 [SMILES: CCC(NC(=O)C1CCN(Cc2ccc3[nH]ccc3c2)CC1)c1nc2ccccc2[nH]1] | ¹H NMR (DMSO-d₆) δ 0.83-0.89(m, 3H), 1.56-1.99(m, 8H), 2.32(s, 1H), 2.85(s, 1H), 3.48(s, 2H), 4.97(d, 1H), 6.36(s, 1H), 6.97 (d, 1H), 7.05(t, 2H), 7.12(d, 2H), 7.30(d, 2H), 7.40(d, 1H), 8.24(d, 1H), 11.01(s, 1H), 12.14(s, 1H) |
| 25 | 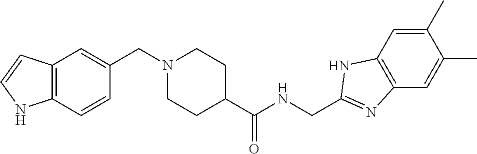 [SMILES: Cc1cc2nc(CNC(=O)C3CCN(Cc4ccc5[nH]ccc5c4)CC3)[nH]c2cc1C] | ¹H NMR (DMSO-d₆) δ 1.17(t, 1H), 1.70(d, 4H), 1.86-1.98(m, 2H), 2.27(d, 8H), 2.88(d, 2H), 3.48(s, 1H), 4.03(q, 1H), 4.41(d, 2H), 6.36(s, 1H), 7.18(d, 1H), 7.28(s, 1H), 7.30 (d, 3H), 7.40(s, 1H), 8.36(t, 1H), 11.01(s, 1H), 11.86(s, 1H) |
| 26 | 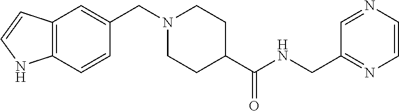 [SMILES: O=C(NCc1cnccn1)C1CCN(Cc2ccc3[nH]ccc3c2)CC1] | ¹H NMR (DMSO-d₆) δ 1.53-1.62(m, 2H), 1.68(d, 2H), 1.73(s, 1H), 1.89(t, 2H), 2.17-2.19(m, 1H), 2.32(s, 1H), 2.72(d, 1H), 2.88(d, 2H), 2.97 (s, 1H), 3.47(s, 9H), 4.38(d, 2H), 6.36(s, 1H), 7.03(d, 1H), 7.30(d, 2H), 7.41(s, 1H), 7.95(s, 1H), 8.50-8.55(m, 3H), 8.56(s, |
| | | 1H), 11.03(s, 1H) |
| 27 | 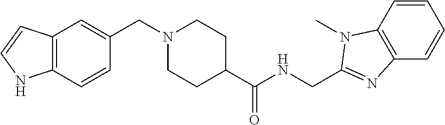 [SMILES: Cn1c(CNC(=O)C2CCN(Cc3ccc4[nH]ccc4c3)CC2)nc2ccccc21] | ¹H NMR( DMSO-d₆) δ 1.17(t, 1H), 1.70(d, 4H), 1.86-1.98(m, 2H), 2.27(d, 2H), 2.88(d, 2H), 3.48(s, 1H), 3.74(s, 3H), 4.03(q, 1H), 4.41(d, 2H), 6.36(s, 1H), 7.18(d, 1H), 7.28(s, 1H), 7.30(d, 3H), 7.40(s, 1H), 8.36(t, 1H), 11.01(s, 1H), 11.86(s, 1H) |
| 28 | 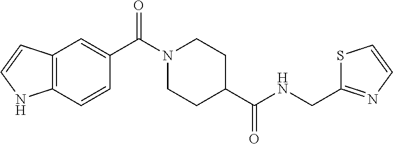 [SMILES: O=C(NCc1nccs1)C1CCN(C(=O)c2ccc3[nH]ccc3c2)CC1] | ¹H NMR (DMSO-d₆) δ 0.92(t, 1H), 1.17(t, 1H), 1.51-1.54(m, 2H), 1.69(s, 2H), 1.98(s, 1H), 2.49-2.50(m, 16H), 2.67-3.17(m, 2H), 3.32(s, 10H), 3.99- 4.11(m, 1H), 4.47(d, 2H), 6.49(t, 1H), 7.12(dd, 1H), 7.40-7.43(m, 2H), 7.58(s, 1H), 7.74(s, 1H), 8.55(t, 1H), 8.95(s, 1H), 11.28(s, 1H) |
| 29 | 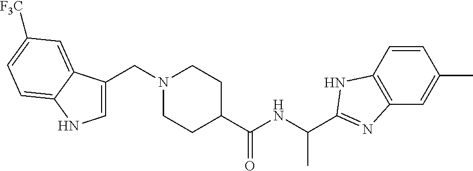 [SMILES: Cc1ccc2[nH]c(C(C)NC(=O)C3CCN(Cc4c[nH]c5ccc(C(F)(F)F)cc45)CC3)nc2c1] | ¹H NMR (DMSO-d₆) δ 1.23(s, 1H), 1.58-1.61(m, 4H), 2.01(t, 2H), 2.32(s, 1H), 2.73(s, 1H), 2.89(s, 2H), 3.17(d, 1H), 3.68(s, 2H), 3.89-4.10(m, 4H), 4.63(s, 1H), 4.82(s, 1H), 7.54(d, 3H), 7.78(s, 1H), 8.31(d, 1H), 11.40(s, 1H), 12.00(s, 1H) |
| 30 | 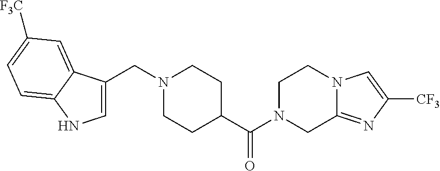 [SMILES: O=C(C1CCN(Cc2c[nH]c3ccc(C(F)(F)F)cc23)CC1)N1CCn2cc(C(F)(F)F)nc2C1] | ¹H NMR (DMSO-d₆) δ 1.23(s, 1H), 1.61(d, 4H), 2.01(t, 2H), 2.32(s, 1H), 2.73(s, 1H), 2.89(s, 2H), 3.17(d, 1H), 3.18(s, 2H), 3.89-4.10(m, 4H), 4.63(s, 1H), 4.82(s, 1H), 7.57(d, 3H), 7.78(s, 1H), 8.01(s, 1H), 11.40(s, 1H) |
| 31 | 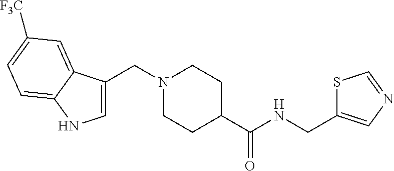 [SMILES: O=C(NCc1cncs1)C1CCN(Cc2c[nH]c3ccc(C(F)(F)F)cc23)CC1] | ¹H NMR (DMSO-d₆) δ 1.23(s, 1H), 1.52-1.61(m, 4H), 2.00-2.08(m, 3H), 2.32(s, 1H), 2.73(s, 1H), 2.89(s, 2H), 3.17(d, 2H), 3.63(s, 2H), 4.10(q, 1H), 4.44(d, 2H), 7.54(d, 3H), 7.71(s, 1H), 8.00(s, 1H), 8.43(t, 1H), 8.93(s, 1H), 11.38(s, 1H) |
| 32 | 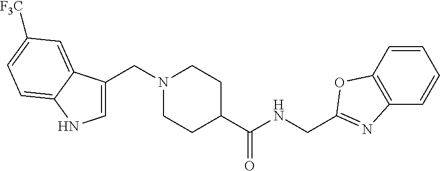 [SMILES: O=C(NCc1nc2ccccc2o1)C1CCN(Cc2c[nH]c3ccc(C(F)(F)F)cc23)CC1] | ¹H NMR (DMSO-d₆) δ 0.87(s, 1H), 1.19(t, 1H), 1.72(d, 4H), 1.90-2.20(m, 4H), 2.74(s, 1H), 2.93(d, 2H), 3.66(s, 2H), 4.05(q, 1H), 4.55(d, 2H), 7.35- 7.38(m, 3H), 7.43(s, 1H), 7.55(d, 1H), 7.69-7.70(m, 2H), 8.02(s, 1H), 8.57(t, 1H), 11.39(s, 1H) |
| 33 | 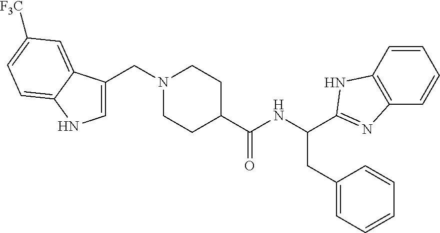 [SMILES: O=C(NC(Cc1ccccc1)c1nc2ccccc2[nH]1)C1CCN(Cc2c[nH]c3ccc(C(F)(F)F)cc23)CC1] | ¹H NMR (DMSO-d₆) δ 1.18-1.29(m, 1H), 1.43-1.50(m, 3H), 1.79(t, 2H), 2.03-2.09(m, 1H), 2.29(t, 1H), 3.12(s, 2H), 3.56(s, 2H), 5.22-5.28(m, 1H), 7.07-7.13(m, 3H), 7.18(d, 4H), 7.30- 7.36(m, 3H), 7.49(d, 2H), 7.94(s, 1H), 7.07-7.11(m, 3H), 7.18(s, 4H), 7.32- 7.34(m, 3H), 7.49(d, 3H), 7.94(s, 1H), 8.45(d, 1H), 11.34(s, 1H) |
| 34 | 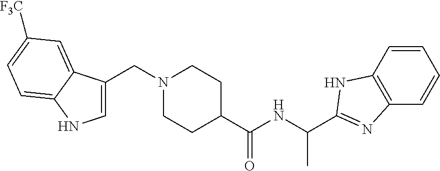 [SMILES: CC(NC(=O)C1CCN(Cc2c[nH]c3ccc(C(F)(F)F)cc23)CC1)c1nc2ccccc2[nH]1] | ¹H NMR (DMSO-d₆) δ 1.23(s, 1H), 1.60(s, 7H), 1.88(t, 2H), 2.18(t, 1H), 2.73(s, 1H), 2.91(s, 2H), 3.17(d, 1H), 3.64(s, 2H), 4.12(d, 1H), 5.09-5.16(m, 1H), 7.13(s, 2H), 7.37(d, 1H), 7.44(s, 2H), 7.54(d, 2H), 8.01(s, 1H), 8.37(d, 1H), 11.39(s, 1H), 12.19(s, 1H) |
| 35 | 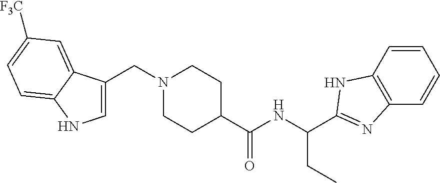 [SMILES: CCC(NC(=O)C1CCN(Cc2c[nH]c3ccc(C(F)(F)F)cc23)CC1)c1nc2ccccc2[nH]1] | ¹H NMR (DMSO-d₆) δ 0.81(t, 3H), 1.50-1.94(m, 9H), 2.28(s, 1H), 2.68(s, 1H), 2.84(s, 2H), 3.11(s, 1H), 3.59(s, 2H), 4.07(q, 1H), 4.89-4.95(m, 1H), 7.07-7.09(m, 2H), 7.49(d, 5H), 7.96(s, 1H), 8.22(d, 1H), 11.34(s, 1H), 12.15(s, 1H) |
| 36 | 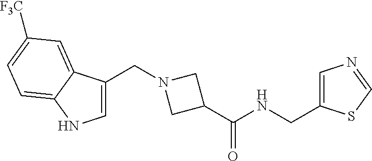 [SMILES: O=C(NCc1cncs1)C1CN(Cc2c[nH]c3ccc(C(F)(F)F)cc23)C1] | ¹H NMR (DMSO-d₆) δ 2.71(s, 1H), 2.88(s, 1H), 3.10-3.13(m, 3H), 3.29- 3.33(m, 2H), 3.67(s, 2H), 4.46(d, 2H), 7.36(s, 1H), 7.41(d, 1H), 7.50(d, 1H), 7.73(s, 1H), 7.92(s, 1H), 8.49(t, 1H), 8.94(s, 1H), 11.36(s, 1H) |
| 37 | 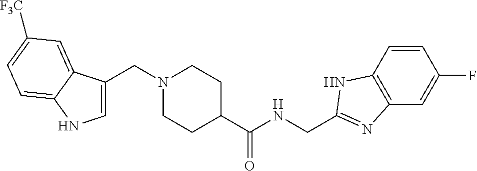 [SMILES: O=C(NCc1nc2cc(F)ccc2[nH]1)C1CCN(Cc2c[nH]c3ccc(C(F)(F)F)cc23)CC1] | ¹H NMR (DMSO-d₆) δ 1.23(s, 1H), 1.54-1.59(m, 2H), 1.71(d, 2H), 1.89(t, 2H), 2.17(t, 1H), 2.32(s, 1H), 2.67(s, 1H), 2.72(s, 1H), 2.92(d, 2H), 3.17(d, 1H), 3.64(s, 2H), 4.43(d, 2H), 6.98(s, 1H), 7.54(d, 5H), 8.01(s, 1H), 8.41(t, 1H), 11.37 (s, 1H), 12.22(s, 1H) |
| 38 | 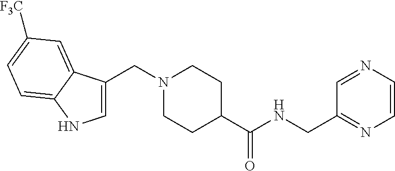 [SMILES: O=C(NCc1cnccn1)C1CCN(Cc2c[nH]c3ccc(C(F)(F)F)cc23)CC1] | ¹H NMR (DMSO-d₆) δ 1.23(s, 1H), 1.53-1.57(m, 2H), 1.67(d, 2H), 1.90(t, 2H), 2.14-2.18(m, 1H), 2.32(d, 1H), 2.90(d, 2H), 3.64(s, 2H), 4.38(d, 2H), 7.36(d, 1H), 7.41(d, 1H), 7.54(d, 1H), 8.00(s, 1H), 8.44(t, 1H), 8.50(t, 2H), 8.56(d, 1H), 11.38(s, 1H) |
| 39 | 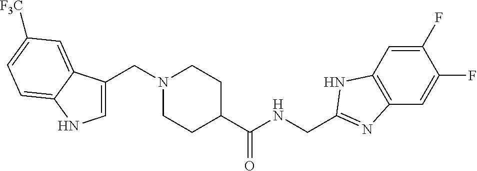 [SMILES: O=C(NCc1nc2cc(F)c(F)cc2[nH]1)C1CCN(Cc2c[nH]c3ccc(C(F)(F)F)cc23)CC1] | ¹H NMR (DMSO-d₆) δ 1.51-1.60(m, 2H), 1.77(s, 2H), 1.89(t, 2H), 2.14- 2.20(m, 1H), 2.72(s, 1H), 2.91(d, 2H), 3.16(s, 1H), 3.64(s, 2H), 4.42(d, 2H), 7.41(d, 2H), 7.52(t, 3H), 8.00(s, 1H), 8.49(t, 1H), 11.38(s, 1H) |
| 40 | 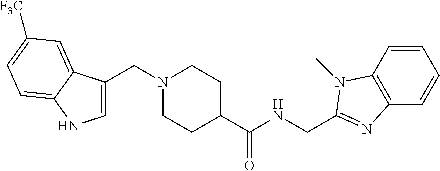 [SMILES: Cn1c(CNC(=O)C2CCN(Cc3c[nH]c4ccc(C(F)(F)F)cc34)CC2)nc2ccccc21] | ¹H NMR (DMSO-d₆) δ 1.48-1.66(m, 4H), 1.84(t, 2H), 2.09-2.15(m, 1H), 2.28(s, 1H), 2.84(t, 2H), 3.13(d, 1H), 3.29(s, 2H), 2.59(s, 2H), 3.68(s, 3H), 4.50(d, 2H), 7.10-7.20(m, 2H), 7.32(d, 1H), 7.36(d, 1H), 7.44-7.53(m, 3H), 7.96(s, 1H), 8.34(t, 1H), 11.35(s, 1H) |
| 41 | 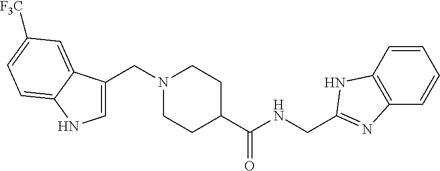 [SMILES: O=C(NCc1nc2ccccc2[nH]1)C1CCN(Cc2c[nH]c3ccc(C(F)(F)F)cc23)CC1] | ¹H NMR (DMSO-d₆) δ 1.69(d, 4H), 1.89(t, 2H), 2.15-2.21(m, 1H), 2.92(d, 2H), 3.64(s, 2H), 4.45(d, 2H), 7.09- 7.14(m, 2H), 7.36(d, 1H), 7.41(s, 1H), 7.46-7.48(m, 2H), 7.54(d, 1H), 7.95(s, 1H), 8.01(s, 1H), 8.46(t, 1H), 11.39(s, 1H) |
| 42 | 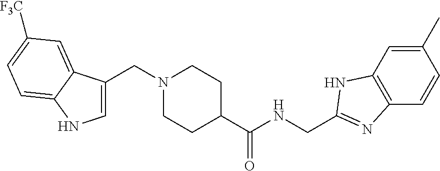 [SMILES: Cc1ccc2nc(CNC(=O)C3CCN(Cc4c[nH]c5ccc(C(F)(F)F)cc45)CC3)[nH]c2c1] | ¹H NMR (DMSO-d₆) δ 1.58(t, 2H), 1.71(d, 2H), 1.89(t, 2H), 2.20(s, 1H), 2.38(s, 3H), 2.91(d, 2H), 3.16(s, 1H), 3.64(s, 2H), 4.10(s, 1H), 4.41(s, 2H), 6.95(d, 1H), 7.25(s, 1H), 7.34-7.41(m, 3H), 7.54(d, 1H), 8.01(d, 1H), 8.42(s, 1H), 11.39(s, 1H), 12.05(s, 1H) |
| 43 | 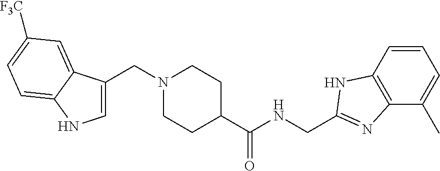 [SMILES: Cc1cccc2[nH]c(CNC(=O)C3CCN(Cc4c[nH]c5ccc(C(F)(F)F)cc45)CC3)nc12] | ¹H NMR (DMSO-d₆) δ 0.01(s, 7H), 1.24(s, 1H), 1.98(t, 6H), 2.20(t, 1H), 2.73(s, 1H), 2.89(d, 2H), 3.21(s, 2H), 3.34(s, 3H), 3.62(s, 2H), 4.10(d, 1H), 4.46(d, 2H), 6.98(s, 1H), 7.02(t, 1H), 7.25(d, 1H), 7.35(d, 1H), 7.42(s, 1H), 7.55(d, 1H), 8.02(s, 1H), 8.43(d, 1H), 11.40(s, 1H), 12.09(s, 1H), 12.30(s, 1H) |
| 44 | 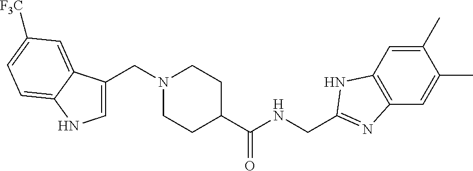 [SMILES: Cc1cc2nc(CNC(=O)C3CCN(Cc4c[nH]c5ccc(C(F)(F)F)cc45)CC3)[nH]c2cc1C] | ¹H NMR (DMSO-d₆) δ 1.51-1.60(m, 2H), 1.70(d, 2H), 1.89(t, 2H), 2.14- 2.17(m, 1H), 2.27(s, 6H), 2.91(d, 2H), 3.17(d, 2H), 3.64(s, 2H), 4.11(q, 1H), 4.40(d, 2H), 6.91-9.99(m, 1H), 7.28(s, 2H), 7.40(s, 2H), 7.54(d, 1H), 8.00(s, 1H), 8.38(t, 1H) |
| 45 | 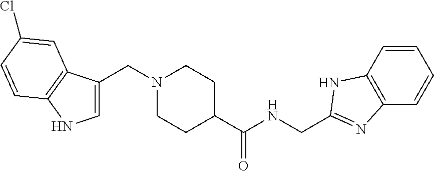 [SMILES: O=C(NCc1nc2ccccc2[nH]1)C1CCN(Cc2c[nH]c3ccc(Cl)cc23)CC1] | ¹H NMR (DMSO-d₆) δ 1.06(s, 1H), 1.23(s, 1H), 1.86(t, 1H), 2.88(s, 3H), 3.16(s, 1H), 3.38 (s, 1H), 3.57(s, 7H), 4.45(d, 1H), 7.05-7.11(m, 3H), 7.44- 7.47(m, 4H), 7.62(s, 1H), 7.94(s, 1H), 8.94(s, 1H), 11.21(s, 1H) |
| 46 | 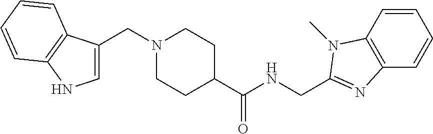 [SMILES: Cn1c(CNC(=O)C2CCN(Cc3c[nH]c4ccccc34)CC2)nc2ccccc21] | ¹H NMR (DMSO-d₆) δ 1.53-1.75(m, 4H), 1.88(t, 2H), 2.13-2.18(m, 1H), 2.50(d, 2H), 3.59(s, 2H), 3.74(s, 3H), 4.53(s, 2H), 6.96(t, 1H), 7.05(t, 1H), 7.14-7.24(m, 3H), 7.34(d, 1H), 7.50(d, 1H), 7.57(d, 1H), 7.63(d, 1H), 8.37(t, 1H) |
| 47 | 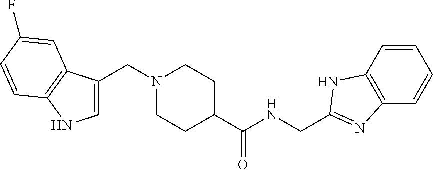 [SMILES: O=C(NCc1nc2ccccc2[nH]1)C1CCN(Cc2c[nH]c3ccc(F)cc23)CC1] | ¹H NMR (MeOH-d₄) δ 1.75-1.91(m, 4H), 2.12(t, 2H), 2.25(s, 1H), 3.03(d, 2H), 3.28(s, 1H), 3.69(s, 2H), 4.56(s, 2H), 6.84(t, 1H), 7.18(s, 2H), 7.26(t, 3H), 7.48(s, 2H) |
| 48 | 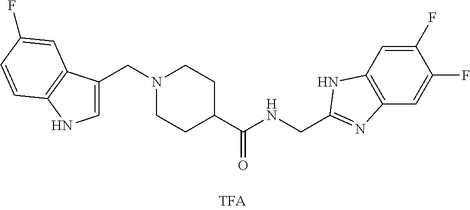 [SMILES: O=C(NCc1nc2cc(F)c(F)cc2[nH]1)C1CCN(Cc2c[nH]c3ccc(F)cc23)CC1] | ¹H NMR (MeOH-d₄) δ 1.84-1.93(m, 1H), 2.13(d, 2H), 2.21(d, 1H), 2.60(t, 1H), 3.05(t, 1H), 3.28-3.37(m, 1H), 3.58(d, 2H), 4.45(s, 2H), 4.70(s, 2H), 6.95(t, 1H), 7.38-7.41(m, 2H), 7.56- 7.62(m, 3H) |
| 49 | 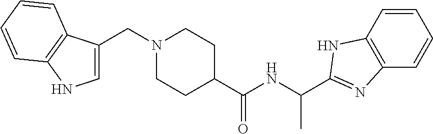 [SMILES: CC(NC(=O)C1CCN(Cc2c[nH]c3ccccc23)CC1)c1nc2ccccc2[nH]1] | ¹H NMR (MeOH-d₄) δ 1.58(d, 3H), 1.72-1.78(m, 4H), 2.04-2.12(m, 2H), 2.18-2.23(m, 1H), 3.00(s, 2H), 3.70(s, 2H), 5.17-5.22(m, 1H), 6.97-7.08(m, 2H), 7.16(t, 3H), 7.33(d, 1H), 7.47- 7.49(m, 2H), 7.59(d, 1H) |
| 50 | 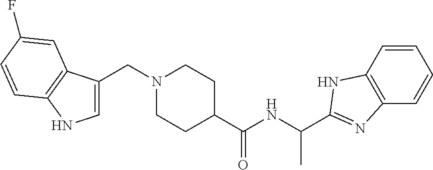 [SMILES: CC(NC(=O)C1CCN(Cc2c[nH]c3ccc(F)cc23)CC1)c1nc2ccccc2[nH]1] | ¹H NMR (MeOH-d₄) δ 1.58(d, 3H), 1.69-1.80(m, 4H), 2.03-2.11(m, 2H), 2.18-2.26(m, 1H), 2.96-3.00(m, 2H), 3.28(s, 1H), 3.65(s, 2H), 5.17-5.23(m, 1H), 6.81-6.68 (m, 1H), 7.14-7.18(m, 2H), 7.22(s, 1H), 7.25-7.28(m, 2H), 7.47-7.49(m, 2H) |
| 51 | 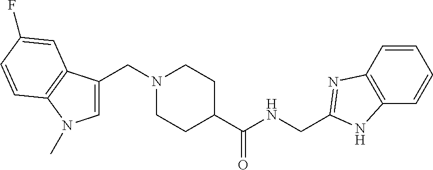 [SMILES: Cn1cc(CN2CCC(C(=O)NCc3nc4ccccc4[nH]3)CC2)c2cc(F)ccc21] | ¹H NMR (CDCl₃) δ 1.78(s, 4H), 2.00(d, 2H), 2.17(s, 1H), 2.97(d, 2H), 3.60(s, 2H), 3.70(s, 3H), 4.58(s, 2H), 6.91- 6.97(m, 2H), 7.14-7.31(m, 4H), 7.52(s, 2H), 7.76(s, 1H) |
| 52 | 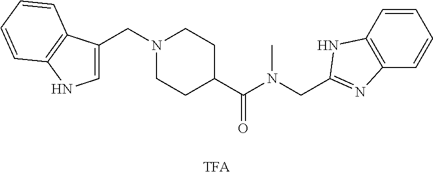 [SMILES: CN(Cc1nc2ccccc2[nH]1)C(=O)C1CCN(Cc2c[nH]c3ccccc23)CC1] | ¹H NMR (MeOH-d₄) δ 1.81-1.91(m, 1H), 2.08(d, 2H), 2.98-3.15(m, 3H), 3.23(s, 1H), 3.28(s, 2H), 3.37(s, 1H), 3.60(d, 2H), 4.47(s, 2H), 4.99(s, 2H), 7.08-7.19(m, 2H), 7.41(d, 1H), 7.49(s, 3H), 7.67-7.71(m, 3H) |
| 53 | 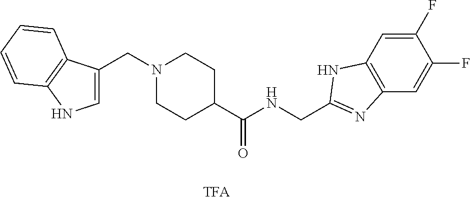 [SMILES: O=C(NCc1nc2cc(F)c(F)cc2[nH]1)C1CCN(Cc2c[nH]c3ccccc23)CC1] | ¹H NMR (MeOH-d₄) δ 1.89(d, 2H), 2.12(d, 2H), 2.25(d, 1H), 2.58(t, 1H), 2.80(s, 0.25H), 3.06(t, 2H), 3.38(s, 0.54H), 3.62(d, 1H), 4.49(s, 2H), 4.68(s, 2H), 7.12-7.20(m, 2H), 7.42(d, 1H), 7.50(s, 1H), 7.56-7.61(m, 2H), 7.69(d, 1H), 11.13(s, 1H) |
| 54 | 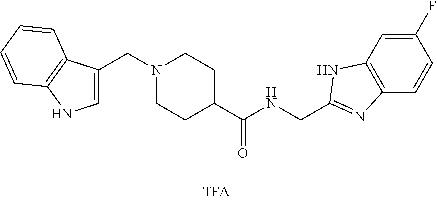 [SMILES: O=C(NCc1nc2ccc(F)cc2[nH]1)C1CCN(Cc2c[nH]c3ccccc23)CC1] | ¹H NMR (MeOH-d₄) δ 1.83-1.93(m, 2H), 2.06(d, 2H), 2.21(d, 1H), 2.58- 2.64(m, 1H), 2.82(s, 1H), 3.06(t, 2H), 3.29(s, 2H), 3.37(s, 1H), 3.62(d, 2H), 4.49(s, 2H), 4.77(s, 2H), 7.08-7.20(m, 2H), 7.26-7.33(m, 1H), 7.39-7.50(m, 3H), 7.63-7.71(m, 2H) |
| 55 | 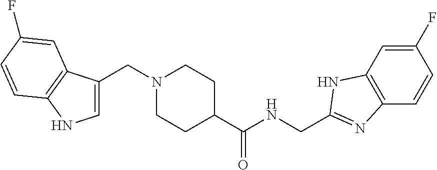 [SMILES: O=C(NCc1nc2ccc(F)cc2[nH]1)C1CCN(Cc2c[nH]c3ccc(F)cc23)CC1] | ¹H NMR (MeOH-d₄) δ 1.70-1.77(m, 4H), 2.08(t, 2H), 2.19-2.26(m, 1H), 3.01(d, 2H), 3.66(s, 2H), 4.54(s, 2H), 6.84(t, 1H), 6.96(t, 1H), 7.19(dd, 1H), 7.23-7.29(m, 3H), 7.45(q, 1H) |
| 56 | 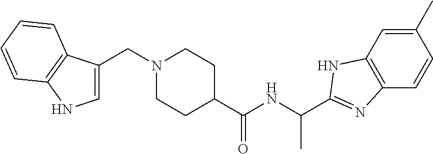 [SMILES: Cc1ccc2nc(C(C)NC(=O)C3CCN(Cc4c[nH]c5ccccc45)CC3)[nH]c2c1] | ¹H NMR (MeOH-d₄) δ 1.59(d, 3H), 1.69-1.78(m, 4H), 2.03-2.10(m, 2H), 2.12-2.19(m, 1H), 2.21(s, 3H), 2.98- 3.02(m, 2H), 3.71(s, 2H), 5.18(q, 1H), 6.99(t, 2H), 7.06(t, 1H), 7.17(s, 1H), 7.27(s, 1H), 7.31-7.37(m, 2H), 7.59(d, 1H) |
| 57 | 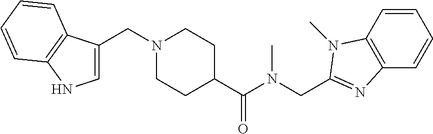 [SMILES: CN(Cc1nc2ccccc2n1C)C(=O)C1CCN(Cc2c[nH]c3ccccc23)CC1] | ¹H NMR (DMSO-d₆) δ 1.61(d, 4H), 1.97(t, 2H), 2.49(s, 2H), 2.58(s, 1H), 2.88(t, 3H), 3.02(s, 2H), 3.61(d, 2H), 3.74(d, 3H), 4.76(s, 1H), 4.90(s, 1H), 6.96(t, 1H), 7.04(t, 1H), 7.15-7.23(m, 3H), 7.31(d, 1H), 7.47-7.62(m, 3H), 8.31(s, 1H), 10.89(s, 1H) |
| 58 | 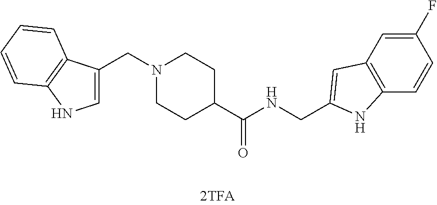 [SMILES: O=C(NCc1cc2cc(F)ccc2[nH]1)C1CCN(Cc2c[nH]c3ccccc23)CC1] | ¹H NMR (MeOH-d₄) δ 1.84-2.30(m, 4H), 2.47(t, 1H), 3.00(t, 2H), 3.37(s, 1H), 3.58(d, 1H), 4.45(s, 4H), 6.24(s, 1H), 6.78(t, 1H), 7.05-7.23(m, 4H), 7.43(d, 1H), 7.49(s, 1H), 7.68(d, 1H), 8.43(s, 1H) |
| 59 | 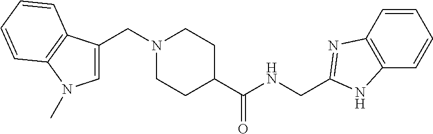 [SMILES: Cn1cc(CN2CCC(C(=O)NCc3nc4ccccc4[nH]3)CC2)c2ccccc21] | ¹H NMR (CDCl₃) δ 1.75-1.81(m, 4H), 2.08(d, 2H), 2.12(s, 2H), 3.01(d, 2H), 3.67(s, 2H), 3.72(s, 3H), 4.56(d, 2H), 6.95(s, 1H), 7.08(t, 1H), 7.16-7.28(m, 5H), 7.52(s, 3H), 7.67(d, 1H) |
| 60 | 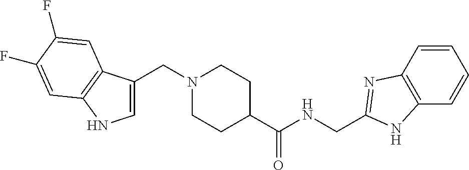 [SMILES: O=C(NCc1nc2ccccc2[nH]1)C1CCN(Cc2c[nH]c3cc(F)c(F)cc23)CC1] | ¹H NMR (MeOH-d₄) δ 1.72-1.88(m, 5H), 2.15(t, 2H), 2.24-2.30(m, 1H), 3.05(d, 2H), 3.28(s, 2H), 3.72(s, 2H), 4.56(s, 2H), 7.16-7.20(m, 3H), 7.24(s, 1H), 7.42-7.48(m, 3H) |
| 61 | 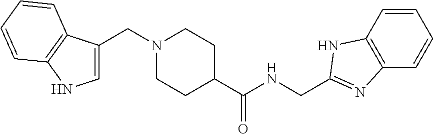 [SMILES: O=C(NCc1nc2ccccc2[nH]1)C1CCN(Cc2c[nH]c3ccccc23)CC1] | ¹H NMR (DMSO-d₆) δ 1.57(d, 4H), 1.69(s, 3H), 1.90(s, 1H), 2.49(s, 1H), 2.68(d, 2H), 2.87(d, 2H), 3.48(s, 2H), 4.45(d, 2H), 6.36(s, 1H), 7.32(d, 3H), 7.40(s, 5H), 7.95(s, 1H), 8.41(t, 1H), 11.01(s, 1H), 12.13(s, 1H) |

### Example 62: 4-((5-Chloro-1H-indol-3-yl)methyl)-N-((5,6-difluoro-1H-benzo[d]imidazol-2-yl)methyl)piperazine-1-carboxamide

### Step 1) Tert-butyl 4-((5-chloro-1H-indol-3-yl)methyl)piperazin-1-carboxylate

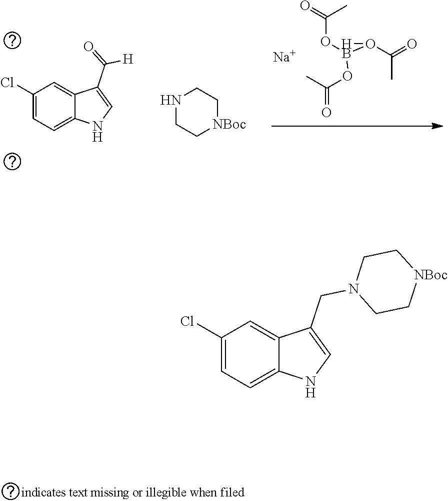

A solution of 5-chloro-1H-indole-3-carbaldehyde (1.000 g, 5.57 mmol) and tert-butyl piperazine-1-carboxylate (1.141 g, 6.12 mmol) in CH₂Cl₂ (27.8 ml) was stirred at room temperature for 1 hour. Sodium triacetoxyborohydrate (1.770 g, 8.35 mmol) was added thereto at once. Then, the reaction mixture was stirred at room temperature for 1 hour, quenched with saturated aqueous NaHCO₃ solution, and extracted with DCM. After concentration in vacuo, the residue was purified by column chromatography on silica gel (SiO₂) (hexane:EtOAc=1:1 to 1:2 to EtOAc only), to obtain tert-butyl 4-((5-chloro-1H-indole-3-yl)methyl)piperazin-1-carboxylate (1.4064 g, 4.02 mmol, 72.2% yield) as a pale yellow solid.

### Step 2) 5-Chloro-3-(piperazin-1-ylmethyl)-1H-indole

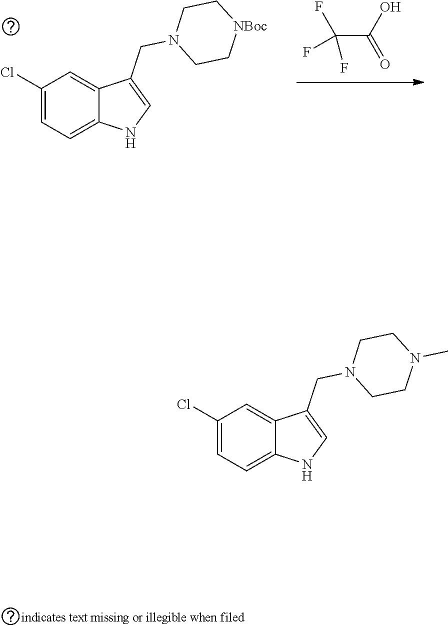 [SMILES: CC(C)(C)OC(=O)N1CCN(Cc2c[nH]c3ccc(Cl)cc23)CC1 ; O=C(O)C(F)(F)F ; CN1CCN(Cc2c[nH]c3ccc(Cl)cc23)CC1]

To a solution of tert-butyl 4-((5-chloro-1H-indol-3-yl)methyl)piperazin-1-carboxylate (0.250 g, 0.715 mmol) in dichloromethane (3.57 ml) was added TFA (2.75 ml, 35.7 mmol) at room temperature. The reaction mixture was stirred at room temperature for 1 hour. After concentration in vacuo, the residue was diluted with toluene and concentrated in vacuo (twice). The residue was quenched with NaBH₄ and extracted with DCM. The combined organic layers were dried over Na₂SO₄, filtered, and concentrated to obtain 5-chloro-3-(piperazin-1-ylmethyl)-1H-indole (0.154 g, 0.617 mmol, 86% yield) as a light yellow solid, which was used in another step without further purification.

### Step 3) 4-((5-Chloro-1H-indol-3-yl)methyl)-N-((5,6-difluoro-1H-benzo[d]imidazol-2-yl)methyl)piperazin-1-carboxamide

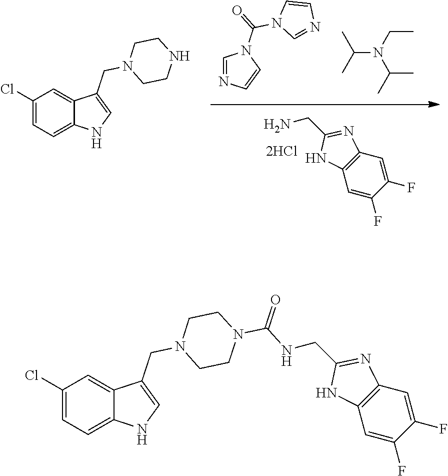

A solution of (5,6-difluoro-1H-benzo[d]imidazol-2-yl)methanamine, 2HCl (0.103 g, 0.400 mmol), di(1H-imidazol-1-yl)methanone (0.062 g, 0.381 mmol), and DIPEA (0.210 ml, 1.201 mmol) in THF (3.81 ml) was stirred at room temperature for 30 minutes. 5-Chloro-3-(piperazin-1-ylmethyl)-1H-indole (0.100 g, 0.400 mmol) was added to the solution, and the reaction mixture was stirred overnight. The reaction mixture was quenched with saturated aqueous NaHCO₃ solution and extracted with EtOAc. The combined organic layers were dried over Na₂SO₄, filtered, and concentrated in vacuo. The residue was purified by column chromatography on silica gel (SiO₂) (EtOAc:MeOH:NH4OH=200:20:1 to 100:10:1), to obtain 4-((5-chloro-1H-indol-3-yl)methyl)-N-((5,6-difluoro-1H-benzo[d]imidazol-2-yl)methyl)piperazine-1-carboxamide (0.085 g, 0.185 mmol, 48.6% yield) as a bright purple solid.

¹H NMR (MeOH-d₄) 2.49 (t, 4H), 3.44 (t, 4H), 3.69 (s, 2H), 4.51 (s, 2H), 7.05 (dd, 1H), 7.23 (s, 1H), 7.30-7.35 (m, 3H), 7.63 (s, 1H)

Compounds of Examples 63 to 77 were synthesized in the same manner as in Example 61, except that appropriate aldehydes are used which correspond to 5-chloro-1H-indole-3-carbaldehyde used in step 1 of Example 62, and adequate amines are used in place of (5,6-difluoro-1H-benzo[d]imidazol-2-yl)methanamine in step 3 of Example 62.

The compounds of Examples 63 to 77 synthesized in the same manner as described above are shown in Table 2.

TABLE 2

| Example | Chemical structure | NMR spectrum data |
|---|---|---|
| 63 | 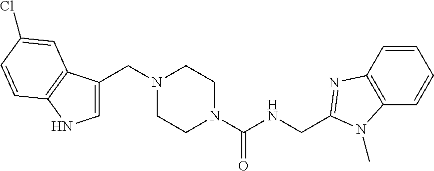 [SMILES: Cn1c(CNC(=O)N2CCN(Cc3c[nH]c4ccc(Cl)cc34)CC2)nc2ccccc21] | ¹H NMR (MeOH-d₄) δ 2.49(s, 4H), 3.43(s, 4H), 3.70(s, 2H), 3.79(s, 3H), 4.60(s, 2H), 7.05(d, 1H), 7.19-7.30(m, 4H), 7.45(d, 1H), 7.57(d, 1H), 7.62(s, 1H) |
| 64 | 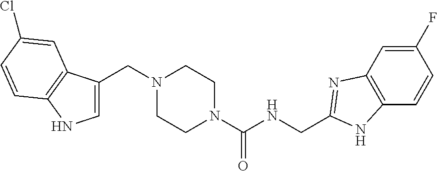 [SMILES: O=C(NCc1nc2cc(F)ccc2[nH]1)N1CCN(Cc2c[nH]c3ccc(Cl)cc23)CC1] | ¹H NMR (MeOH-d₄) δ 2.49(t, 4H), 3.44(t, 4H), 3.69(s, 2H), 4.53(s, 2H), 6.95(t, 1H), 7.06(d, 1H), 7.18(d, 1H), 7.23(s, 1H), 7.30(d, 1H), 7.43(s, 1H), 7.63(s, 1H) |
| 65 | 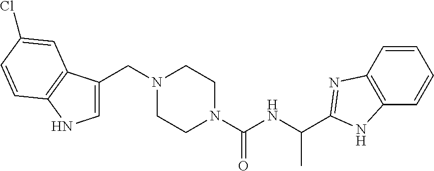 [SMILES: CC(NC(=O)N1CCN(Cc2c[nH]c3ccc(Cl)cc23)CC1)c1nc2ccccc2[nH]1] | ¹H NMR (MeOH-d₄) δ 1.52-1.64(m, 4H), 2.49(s, 4H), 3.49(d, 4H), 3.69(s, 2H), 5.15(q, 1H), 7.05(d, 1H), 7.15-7.19(m, 2H), 7.23(s, 1H), 7.30(d, 1H), 7.48(s, 2H), 7.63(s, 1H) |
| 66 | 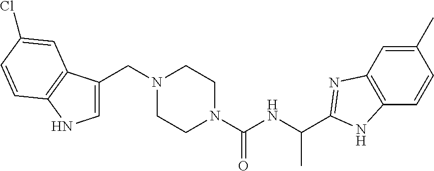 [SMILES: Cc1ccc2[nH]c(C(C)NC(=O)N3CCN(Cc4c[nH]c5ccc(Cl)cc45)CC3)nc2c1] | ¹H NMR (MeOH-d₄) δ 1.51-1.62(m, 3H), 2.41(s, 4H), 2.49(t, 4H), 3.40-3.49(m, 4H), 3.69(s, 1H), 5.12(q, 1H), 7.01(d, 1H), 7.06(dd, 1H), 7.23(s, 1H), 7.29-7.30(m, 2H), 7.36(d, 1H), 7.63(d, 1H) |
| 67 | 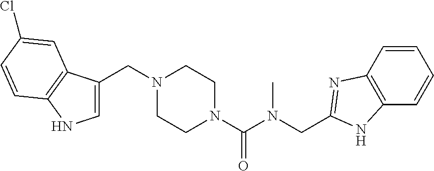 [SMILES: CN(Cc1nc2ccccc2[nH]1)C(=O)N1CCN(Cc2c[nH]c3ccc(Cl)cc23)CC1] | ¹H NMR (MeOH-d₄) δ 2.53(t, 4H), 2.86(s, 3H), 3.35(t, 4H), 3.71(s, 2H), 4.53(s, 2H), 7.05(dd, 1H), 7.18-7.20(m, 2H), 7.23(s, 1H), 7.30(d, 1H), 7.51(s, 1H), 7.63(d, 1H) |
| 68 | 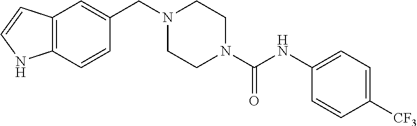 [SMILES: O=C(Nc1ccc(C(F)(F)F)cc1)N1CCN(Cc2ccc3[nH]ccc3c2)CC1] | ¹H NMR (MeOH-d₄) δ 2.50(t, 4H), 3.53(t, 4H), 3.61(s, 2H), 6.39(d, 1H), 7.10(d, 1H), 7.20(d, 1H), 7.34(d, 1H), 7.48-7.54(m, 5H) |
| 69 | 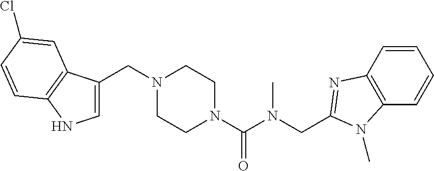 [SMILES: CN(Cc1nc2ccccc2n1C)C(=O)N1CCN(Cc2c[nH]c3ccc(Cl)cc23)CC1] | ¹H NMR (MeOH-d₄) δ 2.52(t, 4H), 2.86(s, 3H), 3.32(t, 4H), 3.71(s, 2H), 3.74(s, 3H), 4.65 (s, 2H), 7.05(dd, 1H), 7.23-7.28(m, 2H), 7.30(d, 2H), 7.46(d, 1H), 7.59(d, 1H), 7.62(d, 1H) |
| 70 | 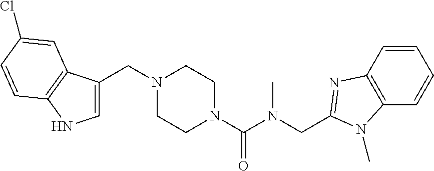 [SMILES: CN(Cc1nc2ccccc2n1C)C(=O)N1CCN(Cc2c[nH]c3ccc(Cl)cc23)CC1] | ¹H NMR (MeOH-d₄) δ 0.89-0.96(m, 3H), 1.86-1.93(m, 1H), 2.01-2.08(m, 1H), 2.40(s, 3H), 2.48(t, 4H), 3.39-3.50(m, 4H), 3.68(s, 2H), 4.92(t, 1H), 7.01(dd, 1H), 7.05(d, 1H), 7.23(s, 1H), 7.27- 7.30(m, 2H), 7.36(d, 1H), 7.63(d, 1H) |
| 71 | 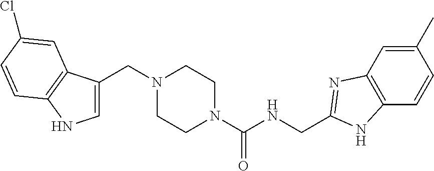 [SMILES: Cc1ccc2[nH]c(CNC(=O)N3CCN(Cc4c[nH]c5ccc(Cl)cc45)CC3)nc2c1] | ¹H NMR (MeOH-d₄) δ 2.40(s, 3H), 2.48(t, 4H), 3.43(t, 4H), 3.68(s, 2H), 4.52(s, 2H), 7.01(d, 1H), 7.05(dd, 1H), 7.23(s, 1H), 7.26(s, 1H), 7.30(d, 1H), 7.35(d, 1H), 7.63(d, 1H) |
| 72 | 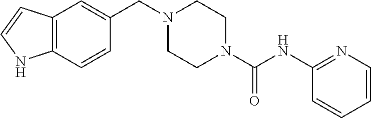 [SMILES: O=C(Nc1ccccn1)N1CCN(Cc2ccc3[nH]ccc3c2)CC1] | ¹H NMR (MeOH-d₄) δ 2.50(t, 4H), 3.54(t, 4H), 3.61(s, 2H), 6.39(d, 1H), 6.97(t, 1H), 7.10(d, 1H), 7.20(d, 1H), 7.34(d, 1H), 7.48(s, 1H), 7.65-7.79(m, 2H), 8.17(d, 1H) |
| 73 | 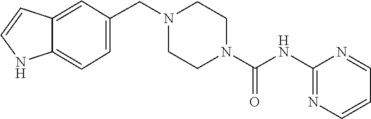 [SMILES: O=C(Nc1ncccn1)N1CCN(Cc2ccc3[nH]ccc3c2)CC1] | ¹H NMR (MeOH-d₄) δ 2.52(t, 4H), 3.57(t, 4H), 3.63(s, 2H), 6.39(d, 1H), 6.99(t, 1H), 7.10(d, 1H), 7.20(d, 1H), 7.33(d, 1H), 7.48(s, 1H), 8.50(d, 2H) |
| 74 | 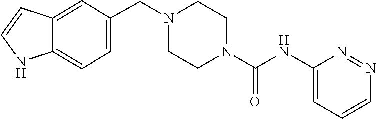 [SMILES: O=C(Nc1cccnn1)N1CCN(Cc2ccc3[nH]ccc3c2)CC1] | ¹H NMR (DMSO-d₆) δ 2.38(t, 4H), 3.49(s, 4H), 3.55(s, 2H), 6.38(s, 1H), 7.06(d, 1H), 7.30-7.35(m, 2H), 7.53(s, 1H), 7.54- 7.56(m, 1H), 7.98(d, 1H), 8.82(d, 1H), 9.06(s, 1H), 11.03(s, 1H) |
| 75 | 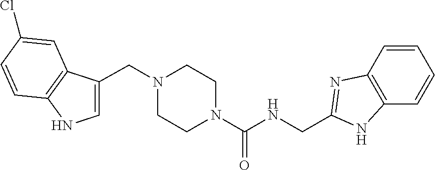 [SMILES: O=C(NCc1nc2ccccc2[nH]1)N1CCN(Cc2c[nH]c3ccc(Cl)cc23)CC1] | ¹H NMR (MeOH-d₄) δ 2.49(t, 4H), 3.44(t, 4H), 3.69(s, 2H), 4.55(s, 2H), 7.05(dd, 1H), 7.15-7.17(m, 2H), 7.23(s, 1H), 7.31(d, 1H), 7.47(s, 2H), 7.63(d, 1H) |
| 76 | 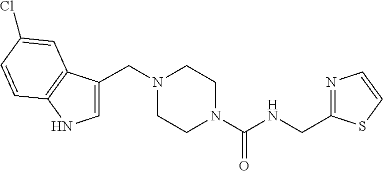 [SMILES: O=C(NCc1nccs1)N1CCN(Cc2c[nH]c3ccc(Cl)cc23)CC1] | ¹H NMR (MeOH-d₄) δ 2.49(t, 4H), 3.43(t, 4H), 3.71(s, 2H), 4.59(s, 2H), 7.05(dd, 1H), 7.24(s, 1H), 7.31(d, 1H), 7.44(d, 1H), 7.63-7.65(m, 2H) |
| 77 | 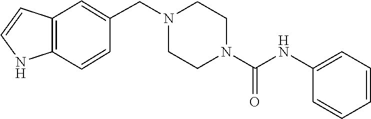 [SMILES: O=C(Nc1ccccc1)N1CCN(Cc2ccc3[nH]ccc3c2)CC1] | ¹H NMR (MeOH-d₄) δ 2.51(t, 4H), 3.51(t, 4H), 3.62(s, 2H), 5.39(d, 1H), 6.98(t, 1H), 7.10(d, 1H), 7.19-7.24(m, 3H), 7.31(t, 3H), 7.48(s, 1H) |

### Example 78: 4-((5-Chloro-1H-indol-3-yl)methyl)-N-(thiazol-2-ylmethyl)piperazin-1-carboxamide

### Step 1) Tert-butyl 4-(thiazol-2-ylmethylcarbamoyl)piperazin-1-carboxylate

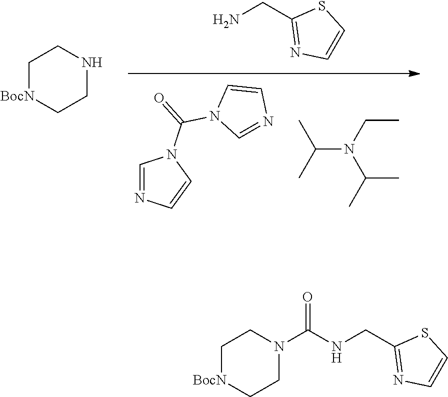

A solution of thiazole-2-ylmethanamine hydrochloride (0.100 g, 0.664 mmol), di(1H-imidazol-1-yl)methanone (0.103 g, 0.632 mmol), and DIPEA (0.168 g, 1.296 mmol) in THF (3.16 ml) was stirred at room temperature for 50 minutes. Tert-butyl piperazine-1-carboxylate (0.124 g, 0.664 mmol) was added to the solution, and the reaction mixture was stirred for 2 hours. The reaction mixture was quenched with saturated aqueous NaHCO₃ solution and extracted with EtOAc. The combined organic layers were dried over Na₂SO₄, filtered, and concentrated in vacuo. The residue was purified by column chromatography on silica gel (SiO₂) (EtOAc:MeOH=50:1), to obtain tert-butyl 4-(thiazol-2-ylmethylcarbamoyl)piperazine-1-carboxylate (0.129 g, 0.395 mmol, 62.5% yield) as a beige solid.

MS (ESI) m/z 326 (M⁺+1)

### Step 2) N-(thiazol-2-ylmethyl)piperazin-1-carboxamide, 2TFA

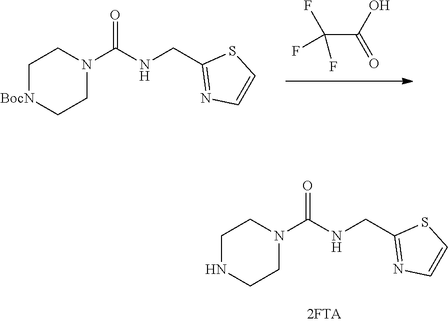 [SMILES: CC(C)(C)OC(=O)N1CCN(C(=O)NCc2nccs2)CC1 ; O=C(O)C(F)(F)F ; O=C(NCc1nccs1)N1CCNCC1]

To a solution of tert-butyl 4-(thiazol-2-ylmethylcarbamoyl)piperazin-1-carboxylate (0.120 g, 0.368 mmol) in DCM (1.838 ml) was added TFA (0.850 ml, 11.03 mmol) at room temperature. The reaction mixture was stirred at room temperature for 1 hour. After concentration under vacuum, a process in which the residue is diluted with toluene and concentrated in vacuo was performed twice, to obtain N-(thiazol-2-ylmethyl)piperazin-1-carboxamide, 2TFA as a yellow oil, which was used in the next step without further purification.

### Step 3) 4-((5-Chloro-1H-indol-3-yl)methyl)-N-(thiazol-2-ylmethyl)piperazin-1-carboxamide

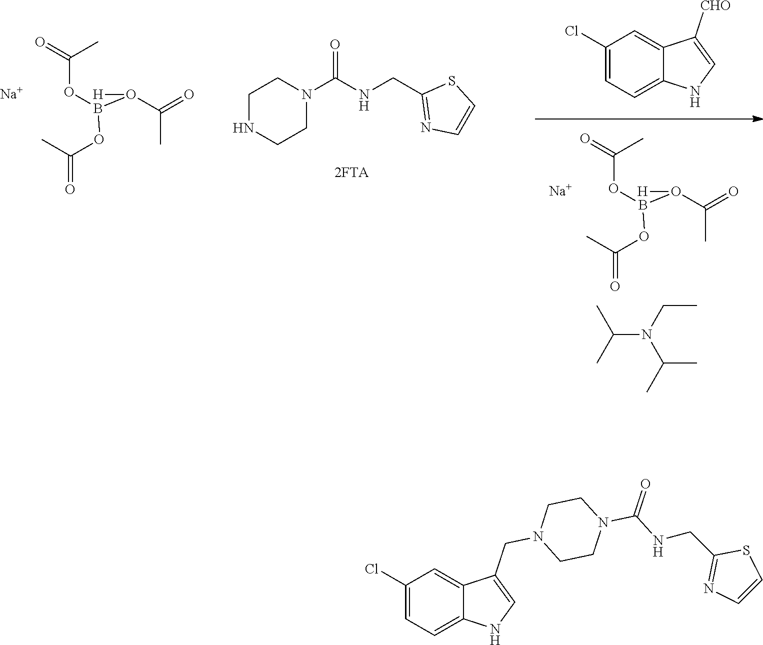

A solution of 5-chloro-1H-indole-3-carbaldehyde (1.000 g, 5.57 mmol), NN-(thiazol-2-ylmethyl)piperazine-1-carboxamide, 2TFA (1.141 g, 6.12 mmol), and triethylamine (2.2 eq) in CH₂Cl₂ (27.8 ml) was stirred at room temperature for 1 hour. Sodium triacetoxyborohydrate (1.770 g, 8.35 mmol) was added thereto at once. Then, the reaction mixture was stirred at room temperature for 1 hour, quenched with saturated aqueous NaHCO₃ solution, and extracted with DCM. After concentration in vacuo, the residue was purified by column chromatography on silica gel (SiO₂) (hexane:EtOAc=1:1 to 1:2 to EtOAc only), to obtain 4-((5-chloro-1H-indole-3-yl)methyl)-N-(thiazol-2-ylmethyl)piperazin-1-carboxamide (71% yield) as a pale yellow solid.

¹H NMR (MeOH-d₄) δ 3.52-3.54 (m, 4H), 3.78 (t, 4H), 4.64 (s, 2H), 7.16 (dd, 1H), 7.41 (d, 1H), 7.47 (d, 1H), 7.67-7.70 (m, 3H)

Compounds of Examples 79 to 89 were synthesized in the same manner as in Example 77, except that appropriate amines are used which correspond to thiazol-2-ylmethaneamine used in step 1 of Example 78, and adequate aldehydes are used in place of 5-chloro-1H-indole-3-carbaldehyde in step 3 of Example 78.

The compounds of Examples 79 to 89 synthesized in the same manner as described above are shown in Table 3.

TABLE 3

| Example | Chemical structure | NMR spectrum data |
|---|---|---|
| 79 | 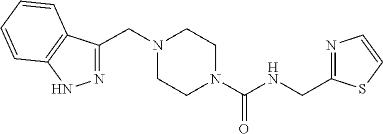 [SMILES: O=C(NCc1nccs1)N1CCN(Cc2n[nH]c3ccccc23)CC1] | ¹H NMR (MeOH-d₄) δ 2.53(t, 4H), 3.43(t, 4H), 3.93(s, 2H), 4.58(s, 2H), 7.12(t, 1H), 7.36(t, 1H), 7.43(d, 1H), 7.48(d, 1H), 7.65(d, 1H), 7.88(d, 1H) |
| 80 | 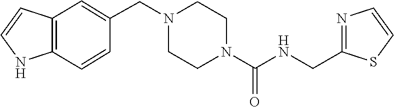 [SMILES: O=C(NCc1nccs1)N1CCN(Cc2ccc3[nH]ccc3c2)CC1] | ¹H NMR (MeOH-d₄) δ 2.47(t, 4H), 3.43(t, 4H), 3.61(s, 2H), 4.59(s, 2H), 6.38(d, 1H), 7.08(d, 1H), 7.20(d, 1H), 7.33(d, 1H), 7.41(d, 1H), 7.48(s, 1H), 7.64(d, 1H) |
| 81 | 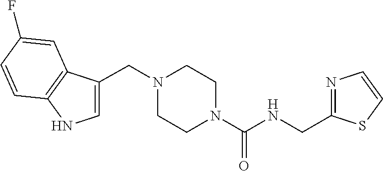 [SMILES: O=C(NCc1nccs1)N1CCN(Cc2c[nH]c3ccc(F)cc23)CC1] | ¹H NMR (MeOH-d₄) δ 2.50(t, 4H), 3.43(t, 4H), 3.70(s, 2H), 4.58(s, 2H), 6.85(t, 1H), 7.24(s, 1H), 7.27-7.31(m, 2H), 7.43(d, 1H), 7.65(d, 1H) |
| 82 | 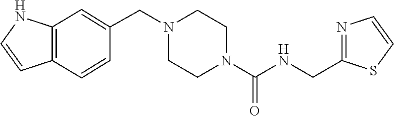 [SMILES: O=C(NCc1nccs1)N1CCN(Cc2ccc3cc[nH]c3c2)CC1] | ¹H NMR (MeOH-d₄) δ 2.47(t, 4H), 3.43(t, 4H), 3.62(s, 2H), 4.59(s, 2H), 6.38(d, 1H), 6.99(d, 1H), 7.18(d, 1H), 7.33(s, 1H), 7.41(d, 1H), 7.48(d, 1H), 7.64(d, 1H) |
| 83 | 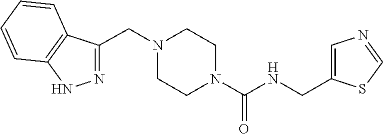 [SMILES: O=C(NCc1cncs1)N1CCN(Cc2n[nH]c3ccccc23)CC1] | ¹H NMR (MeOH-d₄) δ 2.49(t, 4H), 3.38(t, 4H), 3.91(s, 2H), 4.50(s, 2H), 7.11(t, 1H), 7.35(t, 1H), 7.49(d, 1H), 7.69(s, 1H), 7.87(d, 1H), 8.83(s, 1H) |
| 84 | 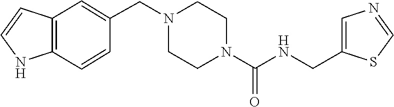 [SMILES: O=C(NCc1cncs1)N1CCN(Cc2ccc3[nH]ccc3c2)CC1] | ¹H NMR (MeOH-d₄) δ 2.44(t, 4H), 3.37(t, 4H), 3.59(s, 2H), 4.50(s, 2H), 6.38(d, 1H), 7.07(d, 1H), 7.20(d, 1H), 7.32(d, 1H), 7.45(s, 1H), 7.70(s, 1H), 8.81(s, 1H) |
| 85 | 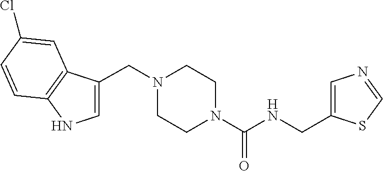 [SMILES: O=C(NCc1cncs1)N1CCN(Cc2c[nH]c3ccc(Cl)cc23)CC1] | ¹H NMR (MeOH-d₄) δ 2.45(t, 4H), 3.38(t, 4H), 3.88(s, 2H), 4.58(s, 2H), 7.05(d, 1H), 7.22(s, 1H), 7.30(d, 1H), 7.61(s, 1H), 7.70(s, 1H), 8.83(s, 1H) |
| 86 | 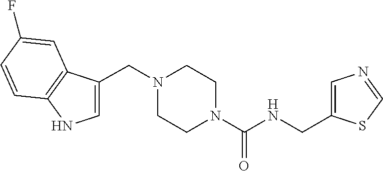 [SMILES: O=C(NCc1cncs1)N1CCN(Cc2c[nH]c3ccc(F)cc23)CC1] | ¹H NMR (MeOH-d₄) δ 2.46(t, 4H), 3.37(t, 4H), 3.69(s, 2H), 4.50(s, 2H), 6.04(t, 1H), 7.22(s, 1H), 7.26-7.29(m, 2H), 7.69(s, 1H), 8.82(s, 1H) |
| 87 | 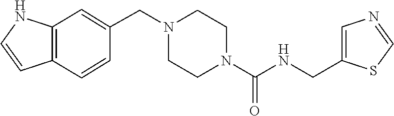 [SMILES: O=C(NCc1cncs1)N1CCN(Cc2ccc3cc[nH]c3c2)CC1] | ¹H NMR (MeOH-d₄) δ 2.43(t, 4H), 3.38(t, 4H), 3.58(s, 2H), 4.50(s, 2H), 6.39(s, 1H), 6.97(d, 1H), 7.19(d, 1H), 7.31(s, 1H), 7.48(d, 1H), 7.70(s, 1H), 8.81(s, 1H) |
| 88 | 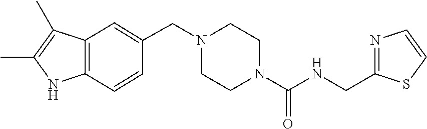 [SMILES: Cc1[nH]c2ccc(CN3CCN(C(=O)NCc4nccs4)CC3)cc2c1C] | ¹H NMR (MeOH-d₄) δ 2.17(s, 3H), 2.31(s, 3H), 2.48(t, 4H), 3.43(t, 4H), 3.61(s, 2H), 4.59(s, 2H), 6.97(d, 1H), 7.16(d, 1H), 7.30(s, 1H), 7.40(d, 1H), 7.65(d, 1H) |
| 89 | 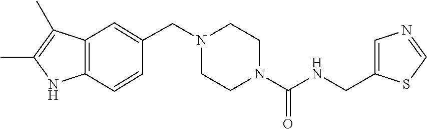 [SMILES: Cc1[nH]c2ccc(CN3CCN(C(=O)NCc4cncs4)CC3)cc2c1C] | ¹H NMR (MeOH-d₄) δ 2.16(s, 3H), 2.30(s, 3H), 2.43(t, 4H), 3.37(t, 4H), 3.88(s, 2H), 4.50(s, 2H), 6.96(d, 1H), 7.15(d, 1H), 7.28(s, 1H), 7.70(s, 1H), 8.80(s, 1H) |

### Test Examples

For the compounds prepared in the above examples, their efficacy was evaluated as follows, and the results are shown.

### Test Example 1: Evaluation of Inhibitory Activity on Binding Between p34 and NEDD4-1 Using Fluorescence Resonance Energy Transfer (FRET) Assay

Inhibitory activity of the compound on binding between p34 and NEDD4-1 was evaluated using p34 protein and NEDD4-1 protein based on the HTRF technology developed by Cisbio. In this assay, a fluorescence resonance energy transfer (FRET) signal was measured in the presence of terbium-labeled anti-FLAG antibody and d2-labeled anti-6XHis antibody. Experiments were performed in 384-well plates under a condition of 1×PBS pH 7.4, 0.1% BSA, and 2% DMSO. A background signal was measured in the absence of the p34-NEDD4-1 proteins, and a non-inhibiting signal was measured with addition of only a solvent (2% DMSO). Then, the compound to be evaluated was applied to give a final concentration of 10 μM. Subsequently, inhibitory activity of the compound on p34-NEDD4-1 was calculated in %. The results are shown in Table 4.

TABLE 4

| Example No. | FRET result (Inhibition % @10 μM) |
|---|---|
| 2 | 42.4 |
| 4 | 30.6 |
| 6 | 36.6 |
| 7 | 59.0 |
| 8 | 49.4 |
| 9 | 58.4 |
| 10 | 39.0 |
| 11 | 39.1 |
| 12 | 39.2 |
| 13 | 38.9 |
| 14 | 44.1 |
| 15 | 60.4 |
| 16 | 47.2 |
| 17 | 48.0 |
| 18 | 30.0 |
| 19 | 32.1 |
| 20 | 51.5 |
| 21 | 56.6 |
| 22 | 48.1 |
| 23 | 38.2 |
| 24 | 53.0 |
| 26 | 42.0 |
| 28 | 34.5 |
| 29 | 59.1 |
| 30 | 59.1 |
| 31 | 58.6 |
| 32 | 61.1 |
| 33 | 41.2 |
| 34 | 59.6 |
| 35 | 45.5 |
| 37 | 39.4 |
| 38 | 36.6 |
| 39 | 37.7 |
| 40 | 42.8 |
| 41 | 42.8 |
| 45 | 33.8 |
| 46 | 42.9 |
| 47 | 30.5 |
| 51 | 56.2 |
| 52 | 32.4 |
| 56 | 56.2 |
| 60 | 38.4 |
| 61 | 52.3 |
| 62 | 30.2 |
| 63 | 52.3 |
| 65 | 53.1 |
| 68 | 49.6 |
| 71 | 33.7 |
| 72 | 55.5 |
| 73 | 53.5 |
| 75 | 52.4 |
| 77 | 35.3 |
| 78 | 61.5 |
| 79 | 54.4 |
| 80 | 49.7 |
| 81 | 50.7 |
| 82 | 47.2 |
| 83 | 41.3 |
| 84 | 44.1 |
| 85 | 44.0 |
| 86 | 41.6 |
| 87 | 48.7 |
| 89 | 31.5 |

### Test Example 2: Evaluation of Activity of p34 Activity Inhibitor Through Induction of PTEN Reactivation

The human colon cancer cell line SW620 was treated with a p34 activity inhibitor at a concentration of 5 μM for 48 hours. Then, the cell solution was collected, and immunoprecipitation with PTEN antibody was performed. Then, the resulting solution was reacted at 37° C. for 40 minutes with a reaction buffer (100 mM Tris-HCl pH 8.0, 10 mM DTT) containing water-soluble diC8-phosphatidylinositol-3,4,5-triphosphate. Subsequently, only the upper solution was collected and reacted with the Biomol Green solution at room temperature for 30 minutes. Lipid phosphatase activity of PTEN was measured at CD650 nm. The phosphatase activity due to PTEN reactivation caused by the compound was calculated in %. The results are shown in Table 5.

TABLE 5

| Example No. | PTEN activity (relative fold @5 μM) |
|---|---|
| 3 | 1.5 |
| 4 | 1.8 |
| 5 | 1.5 |
| 6 | 3.0 |
| 13 | 3.2 |
| 14 | 3.8 |
| 16 | 1.5 |
| 17 | 2.2 |
| 18 | 2.0 |
| 19 | 2.5 |
| 20 | 1.9 |
| 27 | 1.8 |
| 29 | 2.2 |
| 30 | 1.5 |
| 31 | 1.5 |
| 32 | 1.8 |
| 33 | 2.5 |
| 34 | 1.9 |
| 35 | 1.5 |
| 36 | 1.7 |
| 37 | 1.5 |
| 39 | 1.6 |
| 45 | 2.8 |
| 48 | 1.6 |
| 49 | 1.9 |
| 50 | 2.6 |
| 53 | 1.8 |
| 59 | 2.0 |
| 61 | 2.1 |
| 68 | 1.8 |
| 69 | 1.6 |
| 70 | 2.1 |
| 71 | 1.8 |
| 72 | 1.9 |
| 73 | 2.1 |
| 76 | 1.7 |
| 77 | 1.8 |
| 78 | 2.2 |
| 79 | 2.2 |
| 80 | 2.1 |
| 81 | 2.2 |
| 82 | 1.7 |
| 83 | 1.9 |
| 84 | 2.9 |
| 85 | 1.9 |
| 86 | 2.0 |
| 87 | 1.9 |
| 88 | 2.2 |
| 89 | 2.0 |

### Test Example 3: Identification of Binding Site of NEDD4-1 Which Binds to p34

NMR titration was performed to determine a site of the NEDD4-1 protein which binds to p34. 0.5 mM recombinant ¹⁴N-labeled NEDD4-1^{WW1} domain and 0.8 mM unlabeled p34 protein were prepared. Both proteins were prepared by being dissolved in PBS buffer in 10% D₂O containing 137 mM NaCl, 2.7 mM KCl, 8.1 mM Na₂HPO₄, and 1.5 mM KH₂PO₄, pH 7.4. Titration was performed with addition of unlabeled p34 so that the concentration of unlabeled p34 is increased to achieve ratios with ¹⁴N-labeled NEDD4-1^{WW1} domain of 1:0, 1:0.25, 1:0.5. Bruker DRX 850 MHz was used for all NMR experiments, and the results were analyzed by the XWINNMR program and NMRpipe/NMRDraw software.

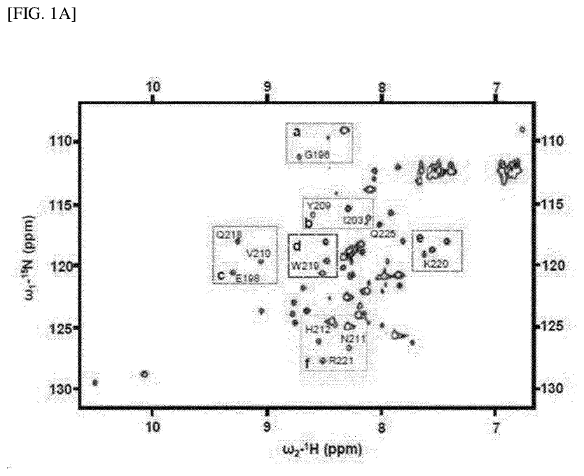

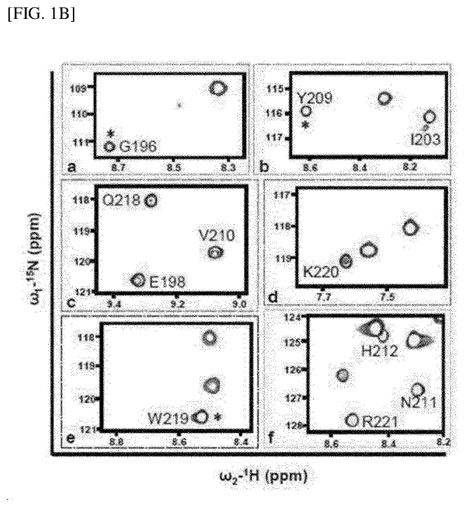

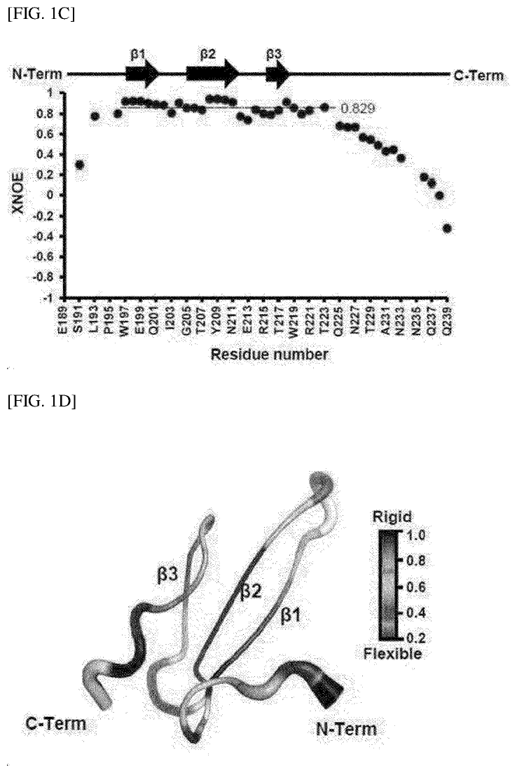

FIGS. 1A to 1D illustrate molecular interactions and backbone dynamics of the WW1 domain of NEDD4-1 after interaction with p34SEI-1. FIG. 1A illustrates superimposed 2D 1H-15N HSQC spectra of the 15N WW1 domain of NEDD4-1 (Red) titrated with increased concentrations of unlabeled p34SEI-1 (1e120). FIG. 1B illustrates an enlarged view of the superimposed 1H-15N HSQC spectra of the WW1 domain of NEDD4-1 titrated with increased concentrations of unlabeled p34SEI-1 (1e120). The asterisk [*] indicates residues that have disappeared due to peak broadening. FIG. 1C illustrates results obtained by performing 1H-15N heteronuclear NOE experiments, the results being expressed in average heteronuclear NOE values for the NEDD4-1 WW1 domain and the corresponding secondary structure (as shown above the panel). FIG. 1D illustrates a depiction in sausage form showing dynamic regions of the NEDD4-1 WW1 domain (SWISS-MODEL).

As illustrated in FIGS. 1A to 1D, the NEDD4-1 protein binds to the p34 protein through the amino acids G196, E198, I203, Y209, V210, N211, H212, K220, and R221 located in the core region of the NEDD4-1 WW1 domain.

Although the present invention has been described with reference to the embodiments, it should be understood that these embodiments are merely illustrative and various modifications and other equivalent embodiments, which are obvious to those skilled in the art, may be implemented within the scope of the present invention as defined in the appended claims.

1. A compound of Formula 1 or a pharmaceutically acceptable salt thereof: 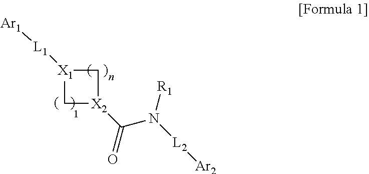
in the formula,
l and n are each independently an integer from 1 to 5,
X₁ and X₂ are each independently N or CH, and at least one thereof is N,
L₁ and L₂ are the same or different from each other and are each independently a single bond, a carbonyl group, an unsubstituted or substituted alkylene group having 1 to 10 carbon atoms, or an unsubstituted or substituted arylene group having 6 to 30 carbon atoms,
Ar₁ and Ar₂ are the same or different from each other and are each independently selected from the group consisting of an unsubstituted or substituted aryl group having 6 to 30 carbon atoms, an unsubstituted or substituted aralkyl group having 6 to 30 carbon atoms, an unsubstituted or substituted heteroaryl group having 3 to 30 carbon atoms, an unsubstituted or substituted heteroarylalkyl group having 6 to 30 carbon atoms, an unsubstituted or substituted cycloalkyl group having 3 to 40 carbon atoms, and an unsubstituted or substituted heterocycloalkyl group having 3 to 40 carbon atoms, and
R₁ is selected from the group consisting of hydrogen, deuterium, a hydroxy group, an alkyl group having 1 to 30 carbon atoms, a cycloalkyl group having 1 to 20 carbon atoms, an alkenyl group having 2 to 30 carbon atoms, an alkynyl group having 2 to 24 carbon atoms, an aralkyl group having 7 to 30 carbon atoms, and an aryl group having 6 to 30 carbon atoms,
wherein the substituted alkylene group, substituted arylene group, substituted heteroarylene group, substituted aryl group, substituted aralkyl group, substituted heteroaryl group, substituted heteroarylalkyl group, substituted cycloalkyl group, and substituted heterocycloalkyl group are those groups each independently obtained by being substituted with at least one substituent selected from the group consisting of hydrogen, deuterium, a cyano group, a nitro group, a halogen group, a hydroxy group, an alkyl group having 1 to 30 carbon atoms, a cycloalkyl group having 1 to 20 carbon atoms, an alkenyl group having 2 to 30 carbon atoms, an alkynyl group having 2 to 24 carbon atoms, an aralkyl group having 7 to 30 carbon atoms, an aryl group having 6 to 30 carbon atoms, a heteroaryl group having 6 to 30 carbon atoms, a heteroaralkyl group having 3 to 30 carbon atoms, an alkoxy group having 1 to 30 carbon atoms, an alkylamino group having 1 to 30 carbon atoms, an arylamino group having 6 to 30 carbon atoms, an aralkylamino group having 6 to 30 carbon atoms, a heteroarylamino group having 6 to 30 carbon atoms, an alkylsilyl group having 1 to 30 carbon atoms, a cycloalkyl group having 3 to 40 carbon atoms, a heterocycloalkyl group having 3 to 40 carbon atoms, an arylsilyl group having 6 to 60 carbon atoms, and an aryloxy group having 6 to 30 carbon atoms; and in a case where those groups are substituted with a plurality of substituents, the substituents are the same or different from each other and may combine with an adjacent group to form a substituted or unsubstituted ring.

2. The compound according to claim 1 or a pharmaceutically acceptable salt thereof,
wherein Ar₁ is selected from the group consisting of compounds represented by Formulas 2 to 4: 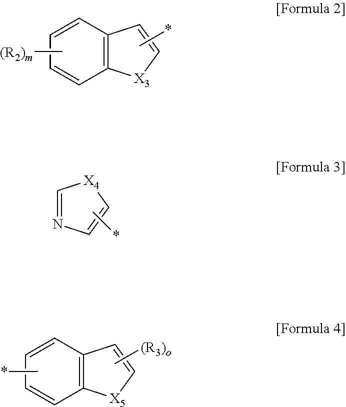
in the formulas,
* means a site where a bond is formed,
m is an integer from 0 to 4,
o is an integer from 0 to 2,
X₃ to X₅ are the same or different from each other and are each independently selected from the group consisting of N(R₄), S, O, and C(R₅)(R₆),
R₂ to R₆ are the same or different from each other and are each independently selected from the group consisting of hydrogen, deuterium, a cyano group, a nitro group, a halogen group, a hydroxy group, an alkyl group having 1 to 30 carbon atoms, a cycloalkyl group having 1 to 20 carbon atoms, an alkenyl group having 2 to 30 carbon atoms, an alkynyl group having 2 to 24 carbon atoms, an aralkyl group having 7 to 30 carbon atoms, an aryl group having 6 to 30 carbon atoms, a heteroaryl group having 6 to 30 carbon atoms, a heteroaralkyl group having 3 to 30 carbon atoms, an alkoxy group having 1 to 30 carbon atoms, an alkylamino group having 1 to 30 carbon atoms, an arylamino group having 6 to 30 carbon atoms, an aralkylamino group having 6 to 30 carbon atoms, a heteroarylamino group having 6 to 30 carbon atoms, an alkylsilyl group having 1 to 30 carbon atoms, a cycloalkyl group having 3 to 40 carbon atoms, a heterocycloalkyl group having 3 to 40 carbon atoms, an arylsilyl group having 6 to 60 carbon atoms, and an aryloxy group having 6 to 30 carbon atoms.

3. The compound according to claim 1 or a pharmaceutically acceptable salt thereof,
wherein Ar₂ is selected from the group consisting of compounds represented by Formulas 5 to 7: 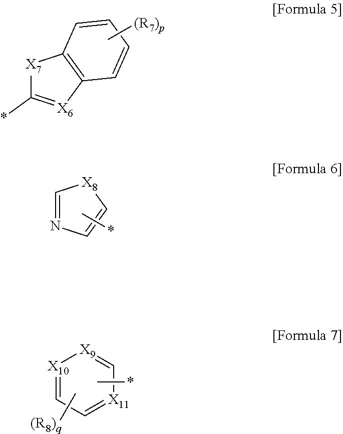
in the formulas,
p is an integer from 0 to 4,
q is an integer from 0 to 2,
X₆ and X₉ to X₁₁ are the same or different from each other and are each independently selected from the group consisting of N, O, S, and C(R₉),
X₇ and X₈ are the same or different from each other and are each independently selected from the group consisting of N(R₁₀), O, S, and C(R₁₁)(R₁₂),
R₆ to R₁₂ are the same or different from each other and are each independently selected from the group consisting of hydrogen, deuterium, a cyano group, a nitro group, a halogen group, a hydroxy group, an alkyl group having 1 to 30 carbon atoms, a cycloalkyl group having 1 to 20 carbon atoms, an alkenyl group having 2 to 30 carbon atoms, an alkynyl group having 2 to 24 carbon atoms, an aralkyl group having 7 to 30 carbon atoms, an aryl group having 6 to 30 carbon atoms, a heteroaryl group having 6 to 30 carbon atoms, a heteroaralkyl group having 3 to 30 carbon atoms, an alkoxy group having 1 to 30 carbon atoms, an alkylamino group having 1 to 30 carbon atoms, an arylamino group having 6 to 30 carbon atoms, an aralkylamino group having 6 to 30 carbon atoms, a heteroarylamino group having 6 to 30 carbon atoms, an alkylsilyl group having 1 to 30 carbon atoms, a cycloalkyl group having 3 to 40 carbon atoms, a heterocycloalkyl group having 3 to 40 carbon atoms, an arylsilyl group having 6 to 60 carbon atoms, and an aryloxy group having 6 to 30 carbon atoms.

4. The compound according to claim 1, or a pharmaceutically acceptable salt thereof,
wherein the compound of Formula 1 is selected from the group consisting of the following compounds:

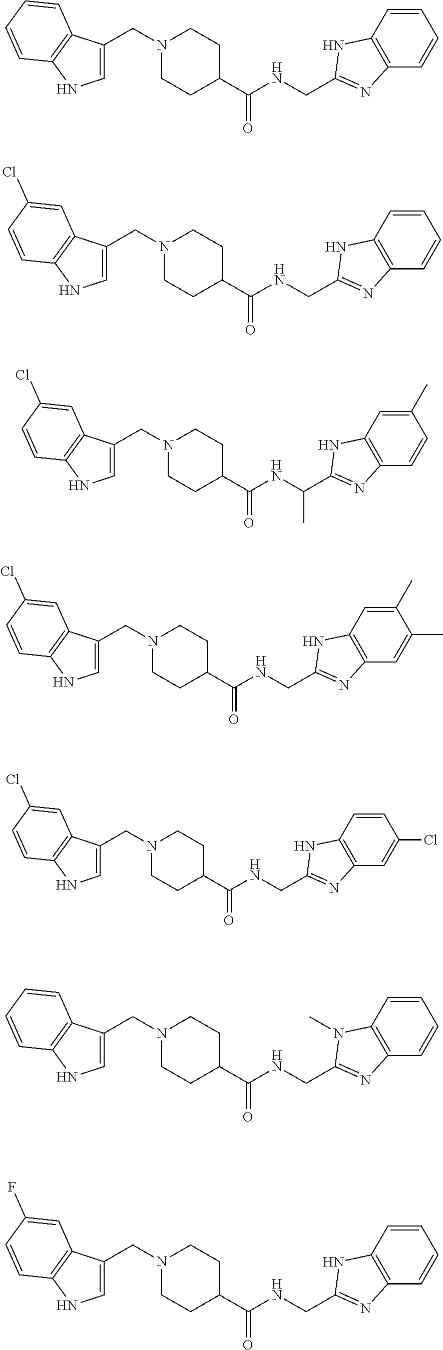

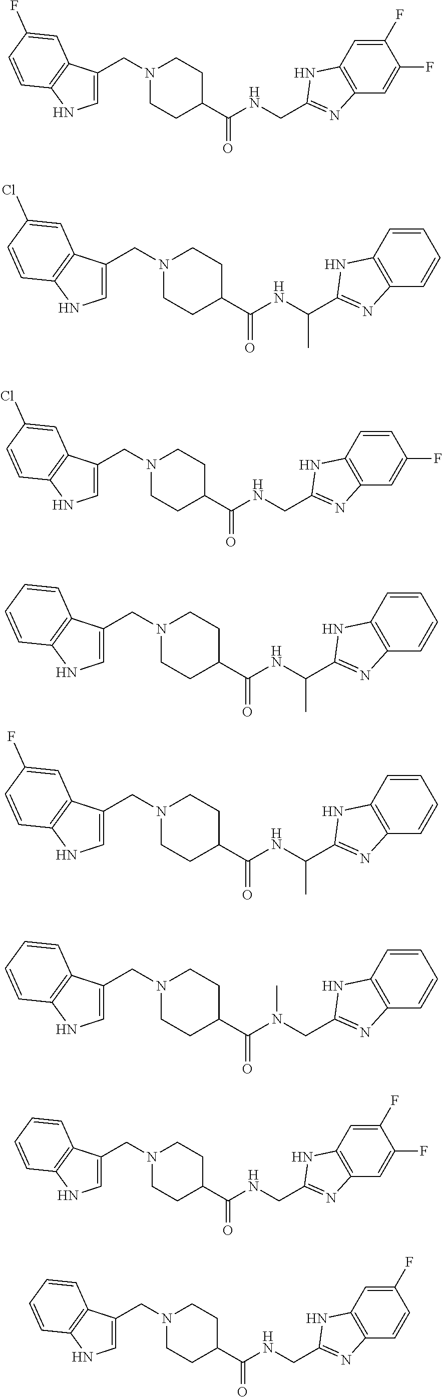

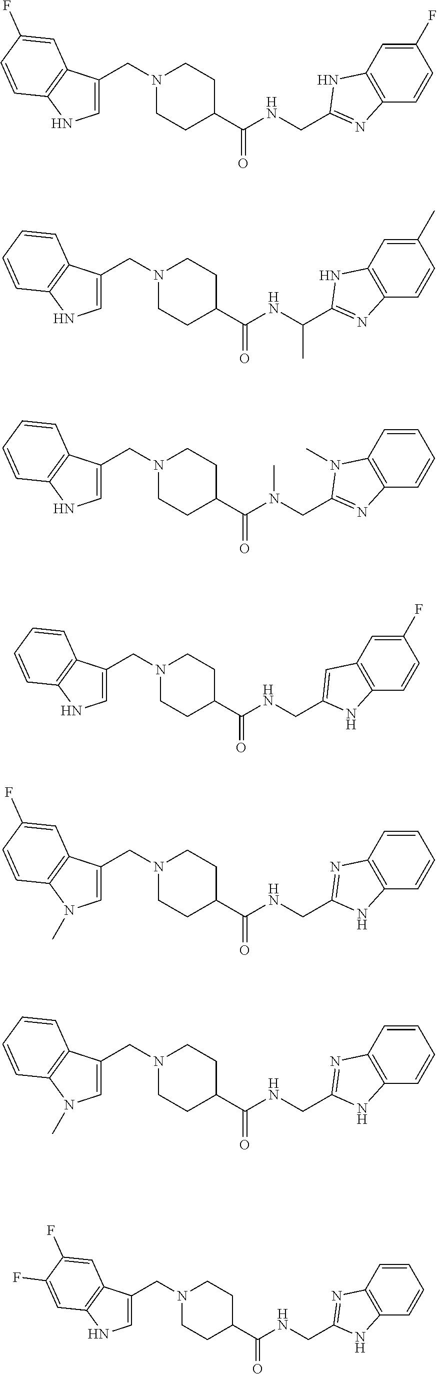

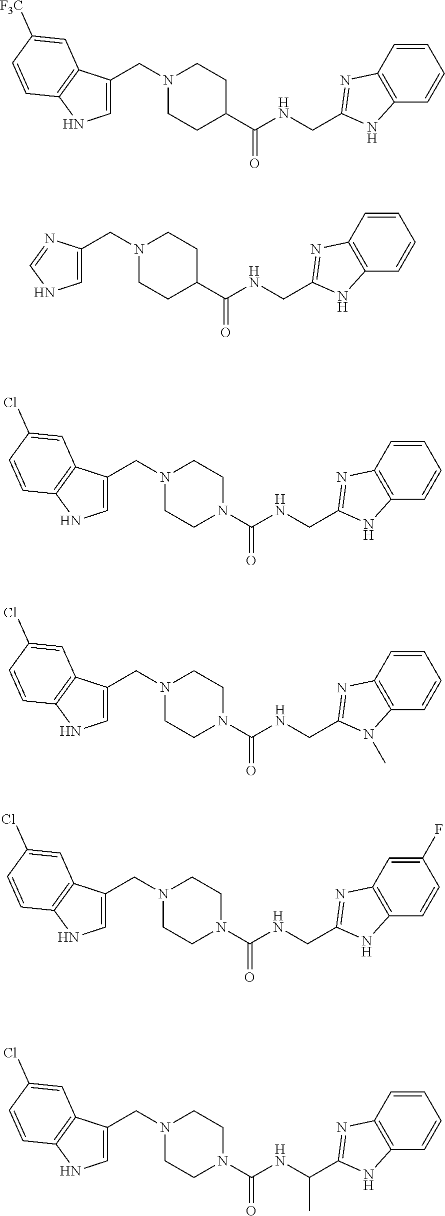 [SMILES: O=C(NCc1nc2ccccc2[nH]1)C1CCN(Cc2c[nH]c3ccc(C(F)(F)F)cc23)CC1 ; O=C(NCc1nc2ccccc2[nH]1)C1CCN(Cc2c[nH]cn2)CC1 ; O=C(NCc1nc2ccccc2[nH]1)N1CCN(Cc2c[nH]c3ccc(Cl)cc23)CC1 ; Cn1c(CNC(=O)N2CCN(Cc3c[nH]c4ccc(Cl)cc34)CC2)nc2ccccc21 ; O=C(NCc1nc2cc(F)ccc2[nH]1)N1CCN(Cc2c[nH]c3ccc(Cl)cc23)CC1 ; CC(NC(=O)N1CCN(Cc2c[nH]c3ccc(Cl)cc23)CC1)c1nc2ccccc2[nH]1]

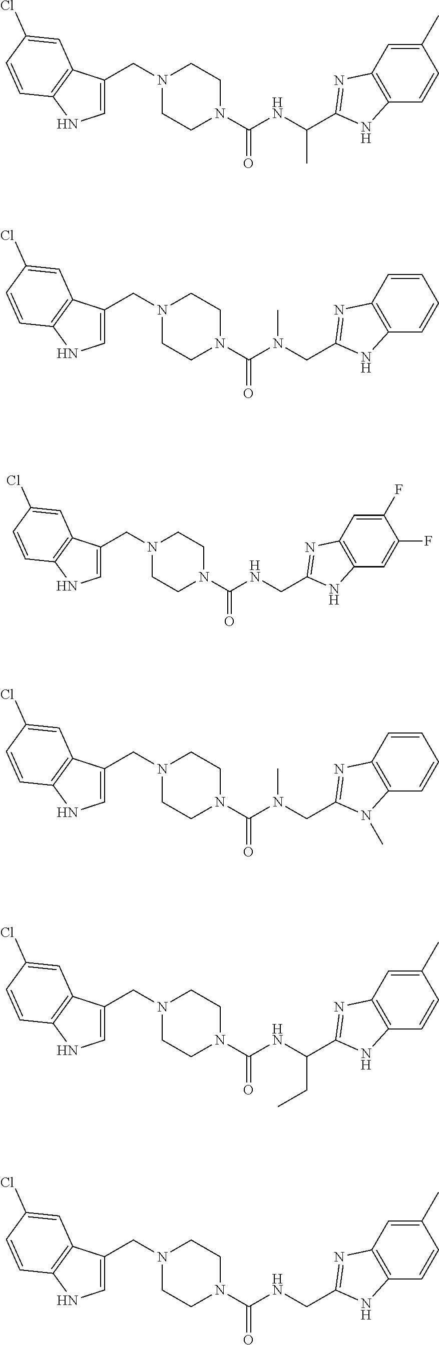 [SMILES: Cc1ccc2[nH]c(C(C)NC(=O)N3CCN(Cc4c[nH]c5ccc(Cl)cc45)CC3)nc2c1 ; CN(Cc1nc2ccccc2[nH]1)C(=O)N1CCN(Cc2c[nH]c3ccc(Cl)cc23)CC1 ; O=C(NCc1nc2cc(F)c(F)cc2[nH]1)N1CCN(Cc2c[nH]c3ccc(Cl)cc23)CC1 ; CN(Cc1nc2ccccc2n1C)C(=O)N1CCN(Cc2c[nH]c3ccc(Cl)cc23)CC1 ; CCC(NC(=O)N1CCN(Cc2c[nH]c3ccc(Cl)cc23)CC1)c1nc2cc(C)ccc2[nH]1 ; Cc1ccc2[nH]c(CNC(=O)N3CCN(Cc4c[nH]c5ccc(Cl)cc45)CC3)nc2c1]

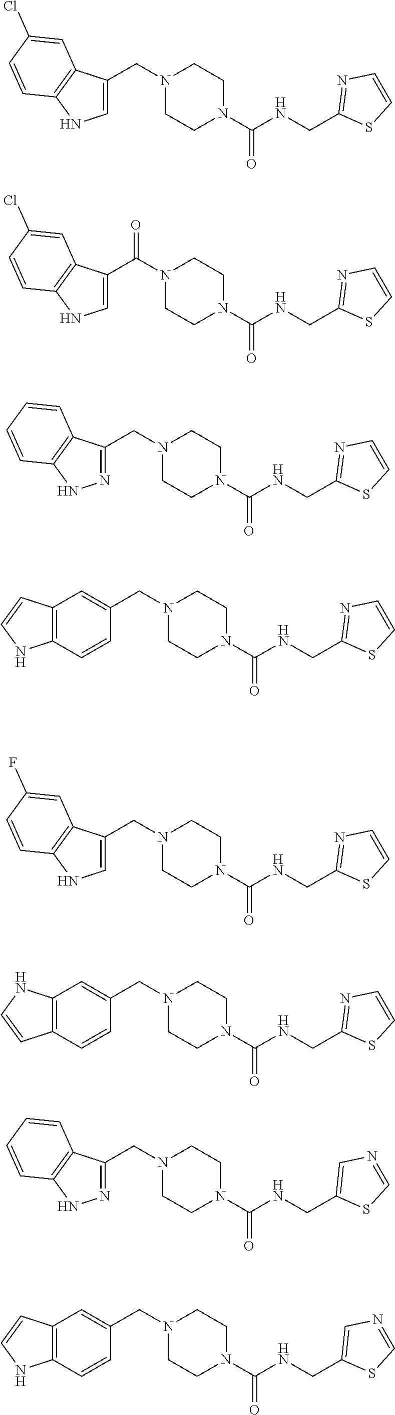

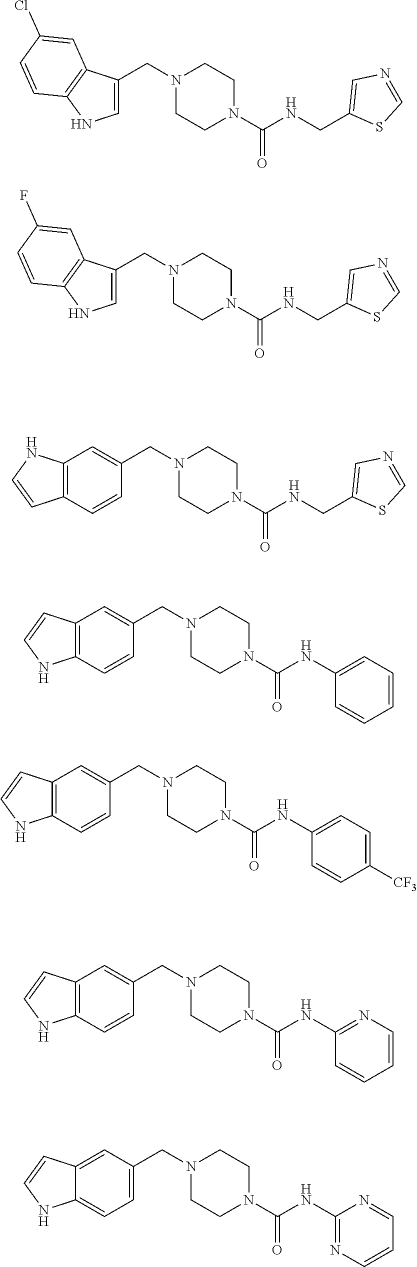

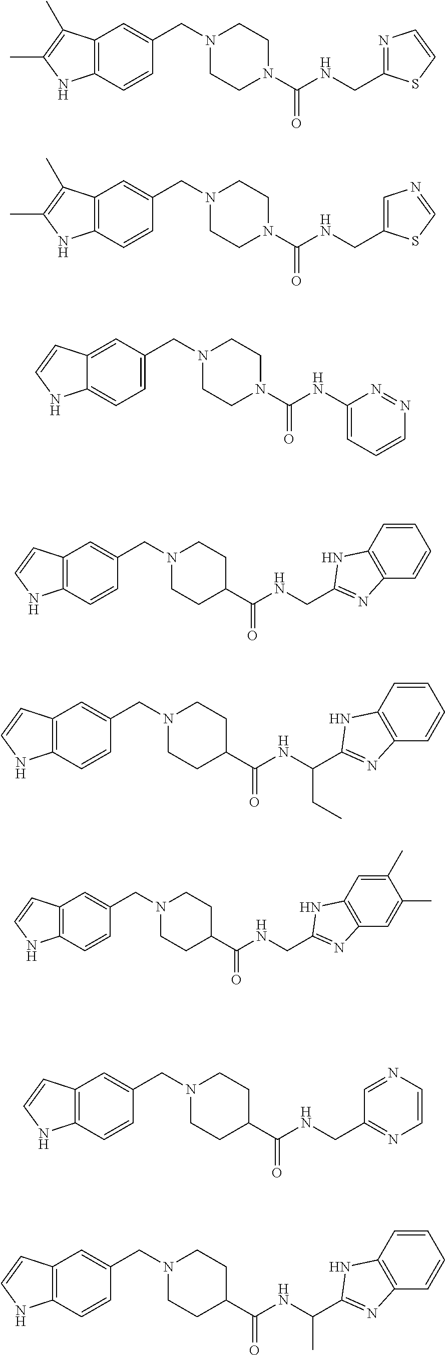

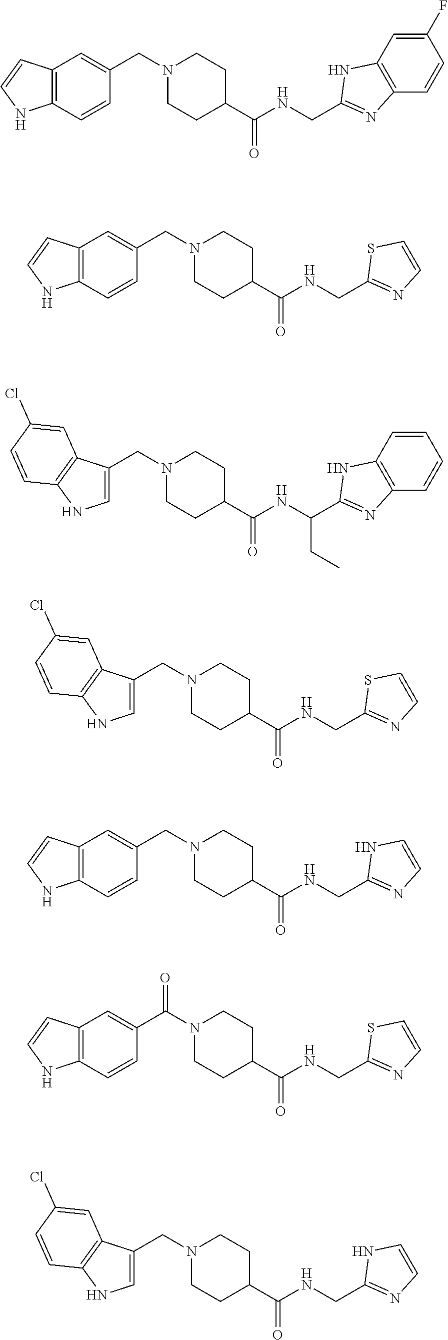

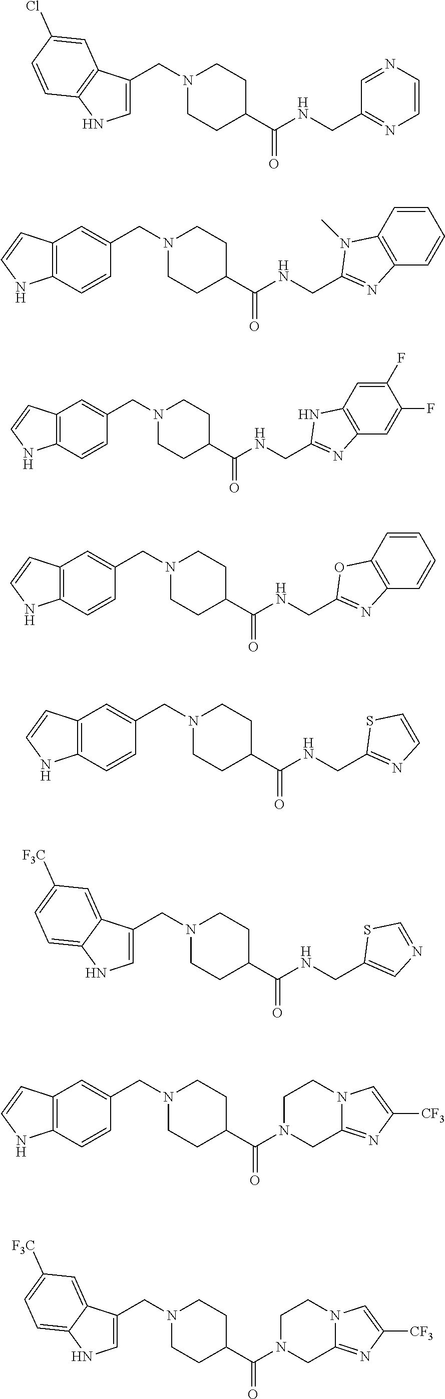

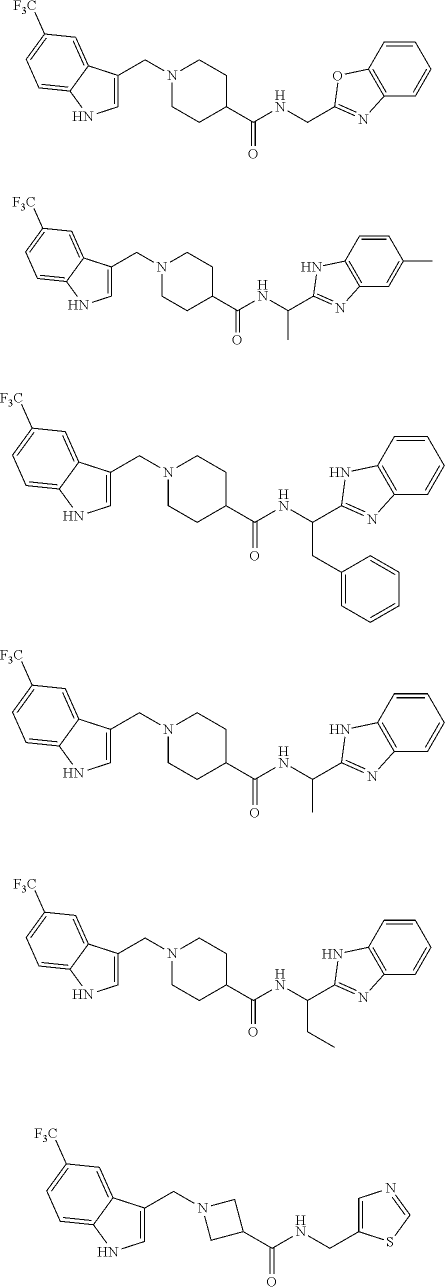 [SMILES: O=C(NCc1nc2ccccc2o1)C1CCN(Cc2c[nH]c3ccc(C(F)(F)F)cc23)CC1 ; Cc1ccc2[nH]c(C(C)NC(=O)C3CCN(Cc4c[nH]c5ccc(C(F)(F)F)cc45)CC3)nc2c1 ; O=C(NC(Cc1ccccc1)c1nc2ccccc2[nH]1)C1CCN(Cc2c[nH]c3ccc(C(F)(F)F)cc23)CC1 ; CC(NC(=O)C1CCN(Cc2c[nH]c3ccc(C(F)(F)F)cc23)CC1)c1nc2ccccc2[nH]1 ; CCC(NC(=O)C1CCN(Cc2c[nH]c3ccc(C(F)(F)F)cc23)CC1)c1nc2ccccc2[nH]1 ; O=C(NCc1cncs1)C1CN(Cc2c[nH]c3ccc(C(F)(F)F)cc23)C1]

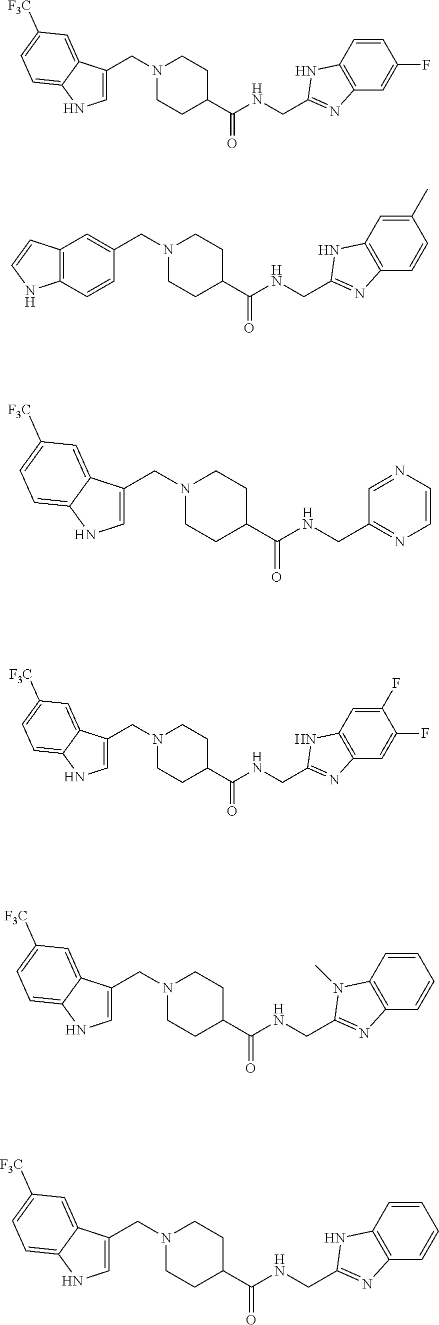 [SMILES: O=C(NCc1nc2cc(F)ccc2[nH]1)C1CCN(Cc2c[nH]c3ccc(C(F)(F)F)cc23)CC1 ; Cc1ccc2nc(CNC(=O)C3CCN(Cc4ccc5[nH]ccc5c4)CC3)[nH]c2c1 ; O=C(NCc1cnccn1)C1CCN(Cc2c[nH]c3ccc(C(F)(F)F)cc23)CC1 ; O=C(NCc1nc2cc(F)c(F)cc2[nH]1)C1CCN(Cc2c[nH]c3ccc(C(F)(F)F)cc23)CC1 ; Cn1c(CNC(=O)C2CCN(Cc3c[nH]c4ccc(C(F)(F)F)cc34)CC2)nc2ccccc21 ; O=C(NCc1nc2ccccc2[nH]1)C1CCN(Cc2c[nH]c3ccc(C(F)(F)F)cc23)CC1]

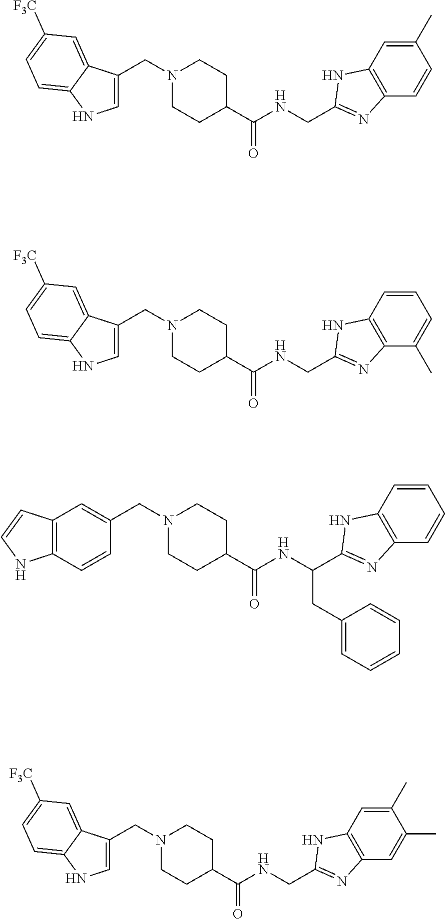 [SMILES: Cc1ccc2nc(CNC(=O)C3CCN(Cc4c[nH]c5ccc(C(F)(F)F)cc45)CC3)[nH]c2c1 ; Cc1cccc2[nH]c(CNC(=O)C3CCN(Cc4c[nH]c5ccc(C(F)(F)F)cc45)CC3)nc12 ; O=C(NC(Cc1ccccc1)c1nc2ccccc2[nH]1)C1CCN(Cc2ccc3[nH]ccc3c2)CC1 ; Cc1cc2nc(CNC(=O)C3CCN(Cc4c[nH]c5ccc(C(F)(F)F)cc45)CC3)[nH]c2cc1C]

5. A pharmaceutical composition, comprising:
a pharmaceutically effective amount of the compound according to claim 1 or a pharmaceutically acceptable salt thereof.

6. A pharmaceutical composition, comprising:
a pharmaceutically effective amount of the compound according to claim 4 or a pharmaceutically acceptable salt thereof.

7. A method for inhibiting binding between p34 protein and neuronal precursor cell-expressed developmentally down-regulated 4-1 (NEDD4-1) protein in a subject or cell, comprising:
a step of administering, to the subject, a pharmaceutically effective amount of the compound according to claim 1.

8. The method according to claim 7,
wherein the compound blocks the NEDD4-1 protein from binding to the p34 protein through the amino acids G196, E198, I203, Y209, V210, N211, H212, K220, and R221 located in the core region of the NEDD4-1 WW1 domain.

9. A method for inhibiting metastasis of cancer cells in a subject, comprising:
a step of administering, to the subject, a pharmaceutically effective amount of the compound of claim 1.

10. A method for treating carcinoma mediated by simultaneous expression of p34 and NEDD4-1, comprising:
a step of administering a pharmaceutically effective amount of the compound of claim 1 to a subject in need of treatment of carcinoma mediated by simultaneous expression of p34 and NEDD4-1.

11. The method according to claim 10,
wherein the carcinoma is selected from the group consisting of carcinomas of lung, breast, colorectum, colon, liver, biliary tract, gastrointestinal tract, head and neck, pancreas, prostate, and cervix, multiple myeloma, melanoma, glioma, and glioblastoma.

12. The method of claim 5 or 6,
wherein the pharmaceutical composition is intended for prevention or treatment of cancer or tumor.

13. The method according to claim 12,
wherein the cancer is selected from the group consisting of liver cancer, hepatocellular carcinoma, thyroid cancer, colon cancer, testicular cancer, bone cancer, oral cancer, basal cell carcinoma, ovarian cancer, brain tumor, gallbladder carcinoma, biliary tract cancer, head and neck cancer, colorectal cancer, vesical carcinoma, tongue cancer, esophageal cancer, glioma, glioblastoma, renal cancer, malignant melanoma, gastric cancer, breast cancer, sarcoma, pharynx carcinoma, uterine cancer, cervical cancer, prostate cancer, rectal cancer, pancreatic cancer, lung cancer, skin cancer, colorectal cancer, colon cancer, and other solid cancers.
